(19) United States
(12) Patent Application Publication
(10) Pub. No.: US 2009/0204101 A1
(43) Pub. Date: Aug. 13, 2009

(54) METHOD OF APPLYING AN INJECTABLE FILLER

(76) Inventors: Mitchell S. Wortzman, Scottsdale, AZ (US); Xiaoming Lin, Cave Creek, AZ (US); Jurrian T. Strobos, Silver Spring, MD (US); Richard G. Glogau, San Francisco, CA (US); Michael A.C. Kane, New York, NY (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/194,934

(22) Filed: Aug. 20, 2008

Related U.S. Application Data
(60) Provisional application No. 60/965,681, filed on Aug. 20, 2007.

(51) Int. Cl. A61M 5/00; G06F 3/048

(52) U.S. Cl. 604/506; 604/93.01; 715/708

(57) ABSTRACT

Methods for applying injectable fillers are provided. In some embodiments, the methods can result in a reduced risk of an adverse event occurring from the administration of the injectable filler. Also disclosed are devices and kits related to this method.

### RELATED APPLICATIONS

This application claims priority to U.S. Provisional Application Ser. No. 60/965,681, filed Aug. 20, 2007, hereby incorporated by reference in its entirety.

### FIELD OF THE INVENTION

Methods and systems for using injectable fillers are disclosed. In particular, methods and systems are provided for using injectable dermal fillers that reduce the risk of an adverse event occurring in a subject undergoing a cosmetic treatment.

### BACKGROUND OF THE INVENTION

### Description of the Related Art

A variety of methods and substances exist for adding volume or firmness to a subject or subject's face for cosmetic purposes. Despite the fact that such methods are being used with ever increasing frequency, the art has seen little in the way of developments in regard to certain aspects of these treatment methods.

The market has a number of injectable fillers available to individuals for cosmetic purposes. These injectable fillers provide an excellent alternative to more invasive surgical procedures, such as laser surgery. They also provide an excellent alternative to over-the-counter face creams and chemical peels, which simply may not be as effective as treatment using injectable fillers.

Despite the numerous advantages injectable fillers provide over other cosmetic treatments, the use of injectable fillers has its own problems. In particular, subjects that are treated with injectable fillers often experience relatively minor adverse events that appear to relate to the actual application of the product to the subject. These adverse events often occur immediately upon treatment and can be local to the target area of treatment. Although the type and magnitude of an adverse event varies from subject to subject, it is not uncommon for subjects to experience discomfort during and after treatment using injectable fillers.

The problem of adverse events in patients has been considered by individuals in the medical community (e.g., Lowe et al., “Adverse Reactions to Dermal Fillers: Review,” Dermatol Surg. 31:11, p. 1616-1625 (November 2005)). However, few subsequent studies have further examined the cause of such adverse events, at least in part because of a belief by those of skill in the art that there is little that can be done to prevent or reduce these local adverse events that seem to be an integral part of administering any injectable filler to a subject.

### SUMMARY OF THE INVENTION

In some aspects, the present disclosure provides a method for reducing the likelihood that an adverse event will occur from the administration of an injectable filler.

In some embodiments, the invention comprises a method for reducing the risk that an adverse event will occur in a subject. The method can comprise identifying a subject that will benefit from a reduction in the risk that an adverse event will occur due to the application of an injectable filler and injecting the subject with an injectable filler at a mean injection flow rate of less than 0.8 mL/minute.

In some embodiments, the invention comprises a method for reducing the risk that an adverse event will occur in a subject. The method can comprise applying an injectable filler to a subject, where an administrator of the injectable filler avoids administering the injectable filler at a mean flow rate of more than 0.8 mL/minute. The administrator also avoids using a fanning injection technique.

In some embodiments, the invention comprises a kit for reducing a risk that an adverse event will occur in a subject. The kit can comprise an injectable filler and a set of instructions for administering the injectable filler. The instructions provide that the injectable filler should be injected at a flow rate of no greater than 0.3 mL/minute. The kit can further comprise an injection device for applying the injectable filler to a subject.

In some embodiments, the invention comprises a method for reducing a risk of an adverse event occurring from the administration of an injectable filler. The method can comprise providing instructions that the injectable filler should be applied at an injection rate of 0.8 mL/minute or slower. The instructions are on a computer readable medium.

In some embodiments, the invention comprises a method of distributing an injectable filler. The method can comprise providing an injectable filler to an injectable filler administrator and providing a set of instructions regarding how to apply the injectable filler to the injectable filler administrator. The set of instructions instructs the administrator to inject the injectable filler at a mean flow rate of no more than 0.6 mL/minute.

In some embodiments, the invention comprises a method of reducing a rate of adverse events associated with an injectable filler in a population of subjects. The method can comprise informing one or more injectable filler administrators that the injectable filler is to be injected at a mean flow rate of no more than 0.6 ml/minute. The population of subjects has the injectable filler injected by the one or more injectable filler administrators.

In some embodiments, the invention comprises a method of treating a subject with an injectable filler. The method can comprise identifying a subject that is to receive an injectable filler. The subject desires a reduction in the likelihood of any adverse events occurring from applying the injectable filler. The method further comprises applying the injectable filler to the subject. For the application, the administrator selects a flow rate of the injection of the injectable filler such that a mean flow rate of the injectable filler is less than 0.5 mL/minute for all injections of the injectable filler in a treatment session.

In some embodiments, the invention comprises a method of applying an injectable filler. The method comprises informing a subject to receive an injectable filler of two options. The first option comprises a shorter treatment session and a higher risk of an adverse event. The second option comprises a longer treatment session and a lower risk of an adverse event. The method further comprises the administrator receiving a selection of either the first or the second option from the subject and applying the injectable filler to the subject. If the subject selects the first option, the injectable filler is injected at a mean rate flow that is above 0.6 mL/minute. If the subject selects the second option, the injectable filler is injected at a mean flow rate that is below 0.6 mL/min.

In some embodiments, the invention comprises a method of applying an injectable filler to a subject. The method can comprise injecting a subject with an injectable filler at a first rate, recognizing an adverse event at an injection site on the subject, and injecting the subject with the injectable filler at a second rate. The second rate is slower than the first rate. The second rate is less than 0.8 mL/min.

In some embodiments, the invention comprises a method of applying an injectable filler to a subject. The method can comprise injecting a subject with an injectable filler at a flow rate that is less than 0.8 mL/min and informing the subject of the risk that they will experience an adverse event from the injection of the injectable filler. The risk is no more than a risk associated with an injection of an injectable filler at a flow rate of no more than 0.8 mL/minute.

There is a long-felt need for a method of reducing the likelihood of adverse events associated with the administration of an injectable filler.

These and other embodiments are described in greater detail below.

### BRIEF DESCRIPTION OF THE DRAWINGS

FIG. 1 is a graph depicting the odds of adverse events versus various flow rates.

FIG. 2 provides a flow chart illustrating some embodiments of the present application.

FIG. 3 is a graph depicting acute local reactions to hyaluronic acid dermal fillers as a function of time.

FIGS. 4A-4C illustrate various needle injection techniques.

FIG. 5 is a scatter plot graph depicting adverse events with respect to injection time versus injection volume.

FIG. 6A displays the proportion of a combined study group that experienced an adverse event at a given flow rate.

FIG. 6B displays the number of data points collected for each of the various flow rates.

FIG. 7A displays the proportion of a study group that experienced an adverse event at a given flow rate.

FIG. 7B displays the number of data points collected for each of the various flow rates.

FIG. 8 displays a graph of the odds of an adverse event as a function of flow rate.

While the subject matter of this application can now be described in detail with reference to the figures, it is done so in connection with the illustrative embodiments. It is intended that changes and modifications can be made to the described embodiments without departing from the true scope and spirit of the subject invention as defined in part by the appended claims.

### DETAILED DESCRIPTION OF THE PREFERRED EMBODIMENT

It has now been realized that the standard method of adding volume or firmness to a subject by administering an injectable filler, while adequate for some purposes, has various shortcomings.

It has been discovered that the likelihood that an adverse event, associated with the injection of a dermal filler, will occur is correlated with aspects of the dermal filler injection technique that were not previously appreciated in the art. In particular, a faster rate of injection can increase the risk that an adverse event will occur, while a relatively slower rate of injection can decrease the risk that an adverse event will occur. As such, in some embodiments, one or more injections out of an entire treatment session on an individual can employ this slower injection technique in order to reduce the risk of adverse events from occurring.

Furthermore, in some embodiments, various specific techniques, such as fanning and the use of larger volumes of the injectable filler, are avoided, which also reduces the likelihood of an adverse event occurring.

The present description first describes various terms used in describing various aspects described herein. A general description of various embodiments of the administration methods is then provided and is followed by a more detailed description of specific aspects of the methods and variations. An additional section regarding additional embodiments is then provided. Finally, examples of using the various methods are disclosed.

The terms “injectable filler composition” and “injectable filler” are used in their ordinary sense as understood by those skilled in the art and thus include a composition that can be administered through injection into or beneath the skin of a subject. The injectable filler composition should not be unduly problematic for the subject receiving the composition. As can be appreciated by one of skill in the art, there are a large number of compositions that can be used as a filler for various embodiments disclosed herein. In some embodiments, the fillers are dermal fillers. In some embodiments, the filler is selected from RESTYLANE® and PERLANE® dermal fillers. Examples of fillers include those disclosed in U.S. Pat. Nos. 5,633,001, 5,007,940, 5,827,937, 5,128,326, 5,399,351, and 5,143,724, as well as PCT Pub. No. WO 87/07898, all of which are herein incorporated by reference in their entireties. In some embodiments, the composition is a cross-linked biocompatible polysaccharide gel composition. In some embodiments, the composition is formed by forming an aqueous solution of a water soluble, cross-linkable polysaccharide; initiating a cross-linking of said polysaccharide in the presence of a polyfunctional cross-linking agent; sterically hindering the cross-linking reaction from being terminating before gelation occurs, an activated polysaccharide thereby being obtained; and reintroducing sterically unhindered conditions for said activated polysaccharide so as to continue the cross-linking thereof up to a viscoelastic gel.

In some embodiments, the injectable filler is characterized by its source. In some embodiments, the source can be biologic and/or synthetic. Biologic injectable fillers can be those that are derived from a living organism. Synthetic injectable fillers can further be divided into two groups, a) man-made fillers for which there is no biologic counterpart and b) man-made substances for which there is a counterpart biologic. In some embodiments, the injectable filler can be characterized by the body's ability to clear a product without external intervention (e.g., these can be biodegradable or nonbiodegradable).

Examples of biologic, biodegradable fillers are those that include materials derived from organism, human, and/or animal tissues and/or products. Examples of such fillers include the following: hyaluronic acid, (such as the following: avian HA, bovine HA, and non-animal stabilized HA (“NASHA”, e.g., RESTYLANE® injectable filler)), collagen (such as collagen I, collagen II, collagen III, cross-linked and/or noncross-linked, bovine, porcine, human, and autologous collagen). Additional examples of collagen based fillers include ZYPLAST® (collagen derived from bovine tissue), ZYDERM® I (collagen derived from bovine tissue), ZYDERM® II, (collagen derived from bovine tissue), EVOLENCE® and EVOLENCE® BREEZE™ (porcine derived collagen), and FIBREL™ (porcine derived collagen). As can be appreciated by one of skill in the art, in some embodiments, the injectable filler is self-replicating, and can include living cells (such as collagen-producing cells or fibroblasts). Thus, in some embodiments there are injectable fillers that are biological and are relatively long lasting or relatively “permanent.”

Synthetic, biodegradable, injectable fillers include RADIANCE™ and RADIESSE™ (microspheres of at least calcium and phosphate ions) injectable fillers, polyacids and polyethers described in U.S. Pat. No. 7,192,984 (e.g., carboxymethyl cellulose (CMC) and polyethylene oxide), and LARESSE® (polymer, polyacid, and/or polyether, similar but not identical to HA type materials).

Synthetic, non-biodegradable, injectable fillers include injectable fillers that are not readily broken down in the body. Synthetic, non-biodegradable, injectable fillers can include injectable fillers that include a biologic component (and vice versa). In some embodiments, at least a portion of product cannot be significantly broken down by various body processes. Additional examples of synthetic non-biodegradable fillers include the following: ARTEFILL™ (polymethylmethacrylate (PMMA) microspheres suspended in bovine collagen), ARTECOL™ (polymethylmethacrylate (PMMA) microspheres suspended in bovine collagen), polymethylmethacrylate (Plexiglas) in bovine collagen carrier, denatured, silicone, and various polymers, polyacids, and polyethers. In some embodiments, the carrier has rapid biodegradation. Of course, as can be appreciated by one of skill in the art, in some embodiments, any one combination, or ingredient of the above fillers can be combined with the other fillers (or alternative fillers) in various embodiments and for particular results.

As can be appreciated by one of skill in the art, injectable fillers need not be categorized by both their source and their ability to stay or be cleared from the body. That is, some fillers can simply be biological, synthetic, biodegradable, or nonbiodegradable. Additionally, as can be appreciated by one of skill in the art, some injectable fillers can include parts or aspects of various combinations of the above or following substances.

Examples of injectable fillers include a substance selected from the following: collagen, fat, human or animal derived collagen, bovine collagen, type I collagen, type II collagen, type III collagen, 3.5% bovine dermal collagen cross-linked by glutaraldehyde to form a latticework, natural human collagen, autologous collagen, polymethylmethacrylate microspheres (optionally suspended in bovine collagen), suspension of collagen fibers prepared from the subject's tissue, human tissue collagen matrix derived from cadaveric dermis, the polyacids and polyethers described in U.S. Pat. No. 7,192,984 (e.g., carboxymethyl cellulose (CMC) and polyethylene oxide), acellular human cadaveric dermis that has been freeze-dried, micronized acellular human cadaveric dermis that has been freeze-dried, cultured autologous fibroblasts, hyaluronic acid, non-animal-stabilized hyaluronic acid derivative, microspheres of calcium hydroxyl appetite suspended in an aqueous gel carrier, dextran beads suspended in hylan gel of nonanimal origin (e.g., 40- to 60-μm in diameter), solubilized elastin peptides with bovine collagen, silicone, solubilized elastin peptides with bovine collagen, poly-L-lactic acid, Gore-Tex (PTFE), glycosylated collagen, PMMA, bone-forming calcium apatite, cultured human cells, expanded polytetrafluoroethylene (e-PTFE), SOFTFORM® of ePTFE, and some combination thereof. Further examples of injectable fillers include the following: AQUAMID® (comprising water and cross-linked polymers), ARTEFILL® (polymethylmethacrylate (PMMA) microspheres suspended in bovine collagen), LARESSE® Dermal Filler (synthetic, biocompatible polymers, non-HA gel comprising absorbable medical polymers), ARTECOLL® (polymethylmethacrylate (PMMA) microspheres suspended in bovine collagen), BELOTERO®, BIO-ALCAMID™ (synthetic reticulate polymer (poly-alkyl-imide), CAPTIQUE™ (non-animal hyaluronic acid), COSMODERM™ (human collagen skin filler), COMOPLAST™, CYMETRA®, autologen, DERMALOGEN®, FASCIAN™ (fascia), fascia, fat, HYLAFORM™ (avian hyaluronic acid), JUVEDERM® (biosynthesized, non-animal hyaluronic acid, including ULTRA or ULTRA PLUS, with and/or without lidocaine or other drug useful for pain relief), RADIESSE™ (microspheres of at least calcium and phosphate ions), SCULPTRA® (poly-L-lactic acid (PLLA)), collagen, hyaluronic acid, RESTYLANE®, PERLANE®, ZYDERM®, ZYPLAST® (collagen derived from bovine tissue), DERMALIVE®, (hyaluronic acid and acrylic hydrogel particles), DERMADEEP® (hyaluronic acid and acrylic hydrogel particles), HYDRAFILL®, ISOLAGEN® (cultured autologous human fibroblasts), LARESSE® (carboxymethylcellulose (CMC) and polyethylene oxide (PEO) filler), PURAGEN™ (filler comprising double cross-linked hyaluron molecules), REVIDERM® INTRA (filler comprising flexible dextran micro-beads suspended in super-coiled, stabilized hyaluronic acid), SCULPTRA™ (formerly NEW-FILL™, filler from poly-L-lactic acid), TEOSYAL®, SURGIDERM® (hyaluronic acid filler involving 3D hyaluronic acid matrix technology), OUTLINE®, ANIKA®, Cosmetic Tissue Augmentation (CTA, from Anika), and combinations thereof.

As can be appreciated by one of skill in the art, any of the above fillers or components thereof can include other materials, for example, anesthetic materials, including, without limitation, lidocaine, prilocaine, tetracaine, etc.

“Volumetric filler” is a type of injectable filler composition. Volumetric fillers can be dermal fillers. In some embodiments, the volumetric filler is capable of crosslinking and/or is cross-linked. Cross-linked compositions allow the filler to have predictably no or minimal volume or substance loss on injection. In some embodiments they also provide predictable expansion or “swelling” with re-hydration on injection: swelling to no more than 10% volume increase; not “shrinking” or losing volume as some fillers that lose water from uncross-linked HA volumes; and/or have sufficient tensile compression resistance. In some embodiments, the volumetric filler involves microbead technology (e.g., as disclosed in U.S. Pat. Nos. 5,633,001 and 5,007,040, herein incorporated by reference in their entireties). In some embodiments, this allows compression resistance. In some embodiments this allows for the composition to have the ability to resist displacement. Other fillers, described as “slurries,” can be used but can be prone to displacement (e.g., disclosed in U.S. Pat. Nos. 5,143,724, 5,633,001, herein incorporated by reference in their entireties). In some embodiments, the filler has the biocompatibility and “feel” of tissue rather than bony implants or sedimentary products that can feel hard. However, bony implant or sedimentary fillers can also be used in some embodiments.

“Dermal filler” is a type of injectable filler composition. Dermal filler denotes that the filler is compatible for use in or under the skin. Dermal fillers can be volumetric fillers. In some embodiments, the dermal filler composition comprises, consists, or consists essentially of a hyaluronic acid or hyaluronic acid derivative. The term “hyaluronic acid” includes salts and bases thereof. In some embodiments, the hyaluronic acid comprises a nonanimal stabilized hyaluronic acid, including gels thereof. In some embodiments, the hyaluronic acid comprises avian HA, bovine HA, or human HA (e.g., RESTYLANE® and PERLANE® injectable fillers)). In some embodiments, the hyaluronic acid comprises at least one of CAPTIQUE™ (non-animal hyaluronic acid), HYLAFORM™ (avian hyaluronic acid), JUVEDERM® (biosynthesized, non-animal hyaluronic acid), DERMALIVE®, (hyaluronic acid and acrylic hydrogel particles), DERMADEEP® (hyaluronic acid and acrylic hydrogel particles), HYDRAFILL®, PURAGEN™ (filler comprising double cross-linked hyaluron molecules), and/or REVIDERM® INTRA (filler comprising flexible DEXTRAN micro-beads suspended in super-coiled, stabilized hyaluronic acid).

The term “improvement” in reference to firmness and/or volume denotes that there has been an increase in the apparent firmness of a subject's skin that has received the injectable filler and/or that there has been an apparent increase in the volume of an under volume area. One example of an increase in volume would include the removal or diminution of lines, wrinkles, and/or undervolume areas in the subject's skin. In some embodiments, an improvement in firmness and/or volume can be described by using the Wrinkle Severity Rating Scale (“WSRS”). Values can be assigned as follows: 1—Absent, 2—Mild, 3—Moderate, 4—Severe, and 5—Extreme. Thus, a decrease in the WSRS can denote an improvement in volume and/or firmness. Of course, the larger the decrease, the larger the improvement in volume and/or firmness. In some embodiments, an improvement in firmness and/or volume can be described by the Global Aesthetic Improvement Scale (“GAIS”), which can have values assigned as follows: 0—Worse; 1—No Change; 2—Improved; 3—Much Improved; and 4—Very Much Improved. In such a scale, the larger the increase, the larger the improvement in volume and/or firmness. Of course, in some embodiments, changes in volume and/or firmness can be characterized simply as a change in volume and/or firmness, without using either the WSRS or the GAIS. The skilled in the art will appreciate that the determination of a WSRS or GAIS score is made by a qualified evaluator.

In some embodiments, the methods described herein are used to alter the appearance of a subject's face. In some embodiments, this alteration is purely an aesthetic alteration. In some embodiments, the alteration does not treat or adjust any deformity that the subject may have. For example, in some embodiments, the subject can simply want added volume to various areas of their face. As such, the application of filler will not necessarily be considered a treatment of the subject's face in all embodiments. Additionally, the term “under volume” does not imply or require that there is necessarily a deformity in the subject's face. Rather, it simply denotes that there appears to be less volume under the skin in one area than in another. In some embodiments, the filler and technique is applied as a treatment of a deformity in a patient. Such applications can be more specifically denoted by the recitation of the fact that a “deformity” is being “treated,” or by the fact that the subject is called a “patient.” Applications in which no deformity is being addressed can be more specifically denoted by the use of the terms “non-treatment,” “subject-preference” or similar term. When such terms are not explicitly used, the techniques and aspects are generic to both treatment and non-treatment applications. As will be appreciated by one of skill in the art, the term “subject” encompasses “patient.” In some embodiments, the method is used to reduce or reverse the signs of aging.

The term “administrator” denotes a human who is qualified to administer an injectable filler to the subject. In some embodiments, the administrator is a doctor or is acting under the guidance of a doctor. In some embodiments, the administrator is not a doctor.

“Target area” as used herein refers to areas or locations to be treated with injectable filler composition, and includes areas or locations that appear to lack volume or are “under volume.” “Target areas” include locations of, for example, oral commissures, marionette lines, mandibular hollows, raise jowls, frowning mouth, pouty lower lip, lateral expression lines, mental creases, chin dimplings, zygomatic hollows, nasolabial folds, tear troughs, malar area or prominence, glabellar lines, crows feet, horizontal forehead lines, peri-oral vertical lines, and brow lifts.

As noted above, the term “treatment” can denote a purely cosmetic result and one that can remove or reduce signs of aging. As will be appreciated by one of skill in the art, a treatment can be performed to achieve a “full correction” of a location. In some situations, this treatment can include a “touch-up” application, approximately one to two weeks after the injection session. As will be appreciated by one of skill in the art, the touch-up application is a step that is done as part of the treatment session and performed to bring the subject's appearance into full correction. This is typically done after the swelling in the subject's face has gone down (due to the initial application of the injectable filler) but before the benefits of the injections are lost. As will be appreciated by one of skill in the art, the touch-up application is designed to bring the treated area into full correction. Thus, in situations where the initial application of the injectable filler brings the area into full correction, no touch-up application is required. In addition, in some embodiments a touch-up application generally involves the application of a smaller amount of an injectable filler compared to the injections of the injectable filler. For example, in some embodiments, the volume of injectable filler applied during the injection session is more than 0.5 cc per side of a subject's face, for example, 0.5-0.6, 0.6-0.7, 0.7-0.8, 0.8-0.9, 0.9-1, 1-1.1, 1.1-1.2 cc or more (e.g., 1-2, 2-3, 3-4, 4-5 cc, or more). The volume of injectable filler applied during the touch-up application is generally less, e.g., approximately 0.2 to 0.3, 0.3-0.4, 0.4-0.5, 0.5-0.6, 0.6-0.7 cc (for each side of a subject's face).

The phrase “injection rate,” “flow rate,” rate of injection,” or other similar term denotes the rate at which the injectable filler enters the subject. When a syringe is used, such a rate can be measured as the rate that the injectable filler leaves the syringe when the syringe is inserted into a subject and is injecting the injectable filler into the subject.

“Adverse event” as used herein refers to a kind of subject reaction that is associated with or occurs as a result of an injection of an injectable filler composition or injectable filler into a subject. A subject reaction can occur because of the intrinsic nature of the filler and/or because of the injection technique and associated needle trauma. Unless explicitly noted otherwise, the term “adverse event” as used herein shall denote a “local” adverse event, which is an adverse event that is located proximal to the injection site.

Adverse events can also be categorized by their time of onset. Adverse events having an “early” onset (up to several days post-treatment) can include injection site reactions, infection, hypersensitivity, lumps caused by maldistribution, discoloration and local tissue necrosis caused by vascular occlusion. Adverse events having a “delayed” onset (occurring weeks to years post-treatment) can include infection, granulomatous inflammation, migration of implants, hypersensitivity, persistent discoloration and persistent scarring.

A “full correction” denotes that the volume desired has been achieved in the subject's skin due to the presence of the injectable filler (e.g., excluding swelling from the application of the injectable fillers).

The terms “odds” is defined by the formula: p/(1−p), where p is the probability of a given event. In the present application, the probability generally refers to the probability of having an adverse event for a given factor (such as a specific flow rate).

The term “mean flow rate” or “mean injection rate” denotes that the mean of the flow rate during the event, such as an injection, is the denoted value. Thus, the flow can be more or less during parts of the injection, as long as, on average, the flow rate is the appropriate value. Unless otherwise stated, “mean flow rate” will refer to the mean flow rate of an injection.

The term “entire injection” in regard to the term flow rate, excludes the very initial start of the injection, as the flow rate at the very start of the injection, before any substantial amount of injectable filler has been administered to the subject, can vary to some extent. Thus, the term “entire injection” denotes the earliest period by which one can obtain the flow rate, all the way to the end of the injection.

While the current use of injectable fillers can provide effective and desirable increases in volume and/or firmness for many subjects, the results are typically associated with one or more adverse events. These adverse events can include bruising, tenderness, edema, pain, erythema, and itch and can cause extreme discomfort to subjects undergoing treatment and immediately thereafter lasting up to two weeks. It is therefore desirable to provide injectable filler treatments whereby the risk of an adverse event occurring is reduced. Prior to the present disclosure, such adverse events appeared to be integrally linked to the injection of the injectable filler itself, and thus, may have been generally assumed to be an inherent aspect of the application of any injectable filler. However, it has been discovered that the risk of an adverse event occurring can be reduced by altering the flow rate of the injection.

In addition, it has been discovered that using a fanning injection technique, rapid injection rate of an injectable filler, and/or a high injection volume of an injectable filler can each result in an increase in adverse events. By applying methods that remove one, two or all three of these factors, it is possible to reduce the likelihood of adverse events occurring and/or the level of discomfort felt by a subject.

In some embodiments, injection techniques that are slow (less than 0.3 ml/min.) along a needle path, and of relatively low volume can be advantageous for reducing adverse events. In some embodiments, the injection technique is applied to the entire area of the subject to be addressed with the dermal filler. In some embodiments, all of the injections meet at least 1, 2, or all 3 of these criteria (e.g., not fanning, low volume, and slow injection). In some embodiments, the dermal filler is administered in a manner so as to avoid adverse events due to 1 or more of the factors identified above that increase the risk of adverse events. In some embodiments, only low volume, slow rate (less than 0.3 mL/min.), and non-fanning techniques are used on a subject.

The technique of employing a relatively slower flow rate during injections can be considered counterintuitive to the manner in which these injectable fillers are generally applied to subjects. In particular, both subjects and administrators generally want the application of the filler to occur in a minimal amount of time, so that the subject is in the least amount of discomfort, and so that the needle is in the subject's skin for the least amount of time. As is appreciated by those of skill in the art, the longer the needle remains in the subject's skin the greater the chance that the needle itself can cause unintended damage in the skin (e.g., scratches). However, in light of the presently disclosed results, it is now clear that there is a significant benefit to be achieved by using a relatively slower rate of injection. Thus, while those of skill in the art may have, prior to the present disclosure, employed ways to increase flow rate, to thereby minimize the amount of time that the needle is in the subject's skin and the duration of the entire treatment session in general, the present disclosure indicates that, counterintuitively, there are times when this is not best for the subject.

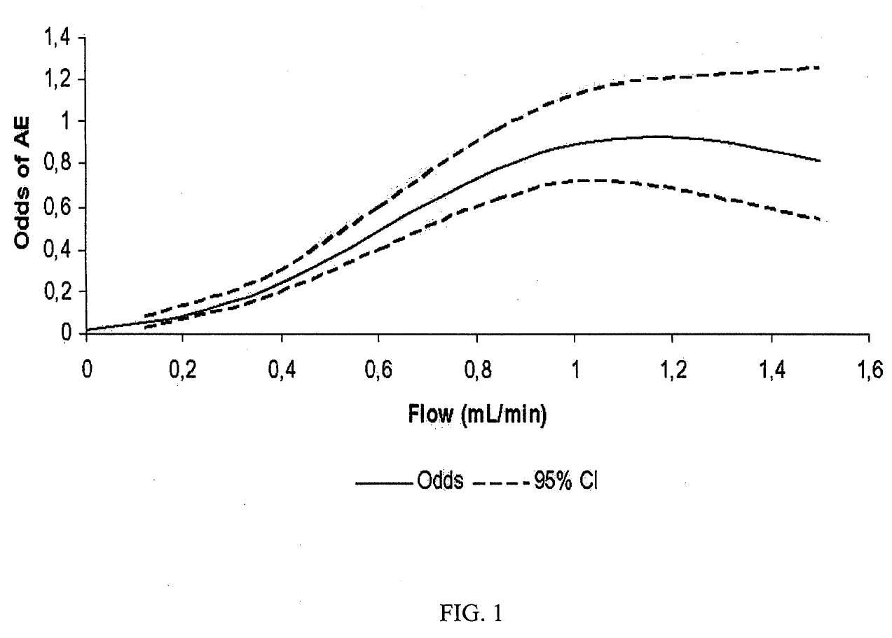

FIG. 1 demonstrates an especially interesting result underlying some of the present embodiments. FIG. 1 is a graph depicting the odds of an adverse event as a function of flow rate. The graph illustrates the unexpected finding that the flow rate of injectable fillers has a strong correlation with the odds of an adverse event occurring in a subject. In particular, the higher the flow rate, the higher the odds of an adverse event occurring. As shown in the graph, keeping the flow rate beneath 1 mL/min. results in a lessening of the odds of an adverse event. As the flow rate decreases, the rate of adverse events also decreases.

The graph in FIG. 1 illustrates results from a study that evaluated the relationship between injectable filler injection techniques and local adverse events. The study was performed on 283 subjects with moderate-to-severe nasolabial folds undergoing treatment with injectable fillers. 142 subjects were injected with nonanimal-stabilized hyaluronic acid gel particles 400 μm in size (NASHA-small, trade name RESTYLANE®), while 141 subjects were injected with nonanimal-stabilized hyaluronic acid gel particles 1,000 μm in size (NASHA-large, trade name PERLANE®). There were no limitations on injection techniques. The subjects were assessed at 72 hours and 2, 6, 12, and 24 weeks for local adverse events. Data collected and examined included physician experience (years), time to correction, volume, depth of injection, number of sessions, and injection techniques. Predefined statistical analysis first identified whether an injection technique variable correlated with an increased rate of local adverse events. If so, sequential logistic regression analysis was completed to assess which predisposing factors had an independent impact on the rate of local adverse events. Surprisingly, and as shown in FIG. 1, the study found that local adverse events were related to injection technique and not to differences in the intrinsic properties of the NASHA agents. Specifically, it was discovered that injection techniques that increase the dissection of the subepidermal plane were shown to increase the incidence of local adverse events. Thus, injection techniques that, if avoided, will reduce the risk of an adverse event include the fanlike needle use, high volume injection, and rapid flow rates (as illustrated in FIG. 1).

As will be appreciated by one of skill in the art, adverse events can occur from both the intrinsic properties of the injectable filler and techniques used to inject the filler into a subject. While certain aspects of the injection technique have been described as influencing adverse reactions (see, e.g., Lowe et al.), it is believed that the present results are the first to credibly demonstrate a strong correlation between the odds of an adverse event and the use of a fanlike needle injection, the injection volume, and/or the flow rate of injectable filler.

In some embodiments, any injection method that reduces the risk of the dissection of the subepidermal plane can provide for a reduced risk of an adverse event occurring. In some embodiments, the method can include relatively slower injection rates, avoiding the use of a fanlike needle injection, and/or using less than 5 mL of injectable filler, such that any dissection of the subepidermal plane is reduced or minimized.

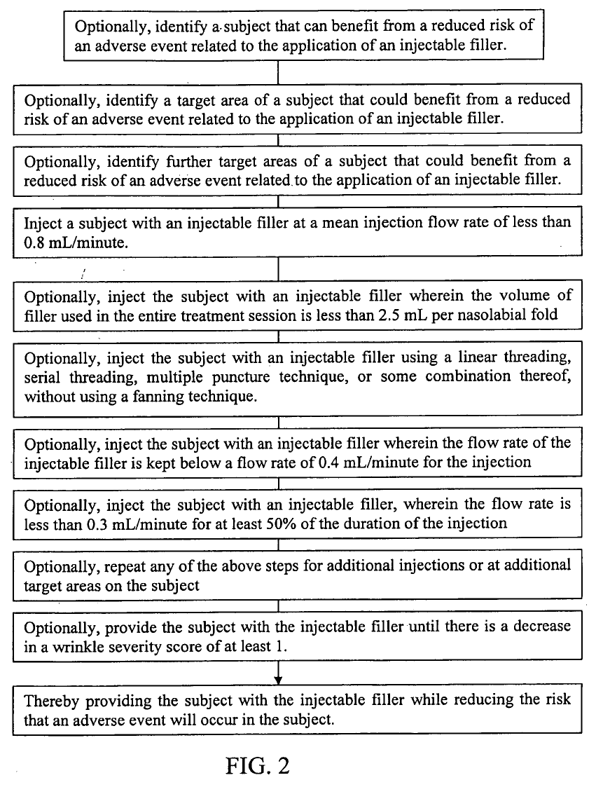

FIG. 2 provides a flow chart illustrating a method according to some embodiments of the present application, including certain optional processes or steps.

In some embodiments, a subject can be injected 40 with an injectable filler at a mean injection flow rate of less than 0.8 mL/minute, thereby providing the subject with the injectable filler (which can be sufficient to achieve a desired result) while reducing the risk that an adverse event will occur in the subject 110.

As shown in FIG. 2 there are additional optional processes that can be employed to advantage for the treatment of a subject receiving an injectable filler.

In some embodiments, the method can start by optionally identifying a subject that can benefit from a reduced risk of an adverse event from the application of an injectable filler 10. In some embodiments, the subject can be selected from a certain class of individuals based on for example, age, gender and/or medical history, that is particularly prone to one or more adverse events when undergoing a treatment using an injectable filler. In other embodiments, a person administering the dermal filler can first identify or recognize a person that will benefit from a reduction in risk of adverse events by subject preference, skin type, subject history, desired result, and/or general knowledge of the person applying the dermal filler.

In some embodiments, the method optionally includes identifying a target area of a subject that could benefit from a reduced risk of an adverse event from a treatment using an injectable filler 20. In some embodiments, target areas can include areas of the subject prone to fine lines, deep lines and wrinkles, oral commissure and other defects, including marionette lines, mandibular hollow, raise jowl, frowning mouth, pout lower lip, later expression lines, mental crease, chin dimpling, zygomatic hollow, nasolabial folds, tear trough, and brow lift.

In some embodiments, this process is optionally repeated so that one can identify multiple target areas of a subject that could benefit from a reduced risk of an adverse event from a treatment using an injectable filler 30. In some embodiments, the subject can benefit from a treatment of multiple target areas using an injectable filler, such as, for example, the lips and forehead. While the same injectable filler can be applied to each target area, in some embodiments, different injectable fillers can be applied. In addition, while the same or similar method of applying the injectable filler can be applied to each target area, in some embodiments, different methods of filler injection can be applied to different target sites to optimize treatment in specific areas. For example, while treatment of one target area can entail utilizing a linear threading technique that injects an injectable filler at a mean flow rate of approximately 0.5 mL/minute per injection, the treatment of another target area can entail utilizing a serial threading technique that injects a filler at a mean flow rate of approximately 0.3 mL/minute per injection.

In some embodiments, the method comprises optionally injecting the subject with an injectable filler but keeping the volume of the filler throughout the entire treatment session at less than 2.5 mL per nasolabial fold 50. In some embodiments, the injection volume is less than 1.04 mL per target area or injection site. In some embodiments, the volume is 0.8 mL or less per target area or injection site. In some embodiments, the volume is less than at least one of the following volumes: 1, 0.9, 0.8, 0.7, 0.6, 0.5, 0.4, 0.3, 0.2, or 0.1 per treatment site. In some embodiments, multiple injections can be applied to a specific target area so long as the total volume of filler is less than 5 mL, e.g., 2, 3, 4, 5, 6, 7, 8, 9, 10 or more injections can be applied to a single treatment site, each injecting a volume of approximately 0.2 mL, such that the entire treatment site receives less than 2 mL. In some embodiments, the method includes this process 50 and process 110, without process 40. Thus, in some embodiments, process 40 is optional.

In some embodiments, the method optionally includes injecting the subject with an injectable filler using a linear technique, serial threading technique, multiple puncture technique or combination thereof, without using a fanning technique in the treatment session 60. One skilled in the art will appreciate that treatment of a target area is not limited to a single technique. In some embodiments, the method includes this process 60 and process 110, without process 40. In some embodiments, process 60 is modified such that it includes any injection technique, as long as it is not a fanning technique.

In some embodiments, the method optionally includes injecting the subject with an injectable filler wherein the flow rate of the injectable filler is kept below a flow rate of 0.4 mL/minute for the injection 70. In other embodiments, one can inject a subject with an injectable filler at an injection flow rate of less than 0.8 mL/minute but not maintain the flow rate at less than 0.8 mL/minute throughout the entire injection (e.g., the flow rate can increase briefly towards the end of an injection to, for example, 1.0 mL/minute). In some embodiments, such as that illustrated by process 70, the injection flow rate can be maintained at less than 0.4 mL/minute throughout the entire injection (or substantially all of the entire injection). In some embodiments, every injection in a treatment session can have the flow rate of the injectable filler below 0.4 mL/minute.

In some embodiments, the method optionally includes injecting the subject with an injectable filler wherein the flow rate of the injectable filler varies such that the flow rate is less than 0.3 mL/minute for at least 50% of the duration of a single injection 80. Thus, in some embodiments, the flow rate can vary between individual injections.

In some embodiments, the method optionally includes repeating any one or more of the above steps for additional injections or at additional target areas on the subject. Any of the steps described in processes 10 through 80 in FIG. 2 can be repeated 1, 2, 3, 4, 5, 6, 7, 8, 9, 10 or more times (e.g., 100 or 200 times in a treatment session) to optimize results.

In some embodiments, the method optionally comprises providing the subject with the injectable filler until it results in a decrease in a Wrinkle Severity Rating Scale (WSRS) of at least 1, 100. Of course, larger improvements in the WSRS can also be achieved.

In addition to the above, it has been discovered that slow, steady injection along the needle path improves acute tolerability of the administration of injectable fillers. Thus in some embodiments, techniques and/or variables identified as favorable above are used for the application of injectable fillers and/or techniques and/or variables identified as increasing risk are avoided for the application of injectable fillers.

In some embodiments, an administrator applies the injectable filler, by a non-fanning technique (e.g., linear threading and/or multiple puncture), slow rate, and/or low volume, because the subject previously experienced adverse events from the application of an injectable filler.

As will be appreciated by one of skill in the art, disclosed herein are at least three different approaches that can be used to avoid or decrease the risk of an adverse event when administering an injectable filler. These approaches include, low flow rate, low volume of injectable filler added, and avoiding techniques such as the fanning technique. For the sake of simplicity, the bulk of the various embodiments, methods, kits, etc., described herein refer to the flow rate embodiment. However, one of skill in the art will appreciate that these various embodiments or aspects also apply to the other two methods as well (unless the embodiment or aspect is only relevant to the rate of flow of the filler); and thus, such embodiments are also contemplated and provided herein.

The following section provides additional alternative embodiments and further specific and exemplary options in regard to the herein disclosed methods.

The methods described herein can reduce the likelihood that one or more adverse event will occur. In some embodiments, the adverse events include at least one of: erythema, edema, pain, tenderness, bruising, itching, acne papule formation, nodule formation, lumping, discoloration (e.g., redness, whiteness and/or hyperpigmentation), and local tissue necrosis caused by vascular occlusion.

In some embodiments, the adverse events include at least one of the following: edema, swelling, bruising, tenderness, erythema, pain, itching, acne papule formation, nodule formation, lumping, discoloration (e.g., redness, whiteness, hyperpigmentation), and local tissue necrosis caused by vascular occlusion. In some embodiments, the adverse event is selected from the group consisting of at least one of the following: bruising, redness, swelling, pain, tenderness, itching, pimples, sore throat, and runny nose, firmness, lumps/bumps, discoloration, nodule formation, erythema, pruritus, desquamation, ecchymosis, edema, granuloma, contour irregularities, numbness, dryness, peeling, burning sensation, whiteheads, rash, and some combination thereof.

In some embodiments, the adverse event is a local adverse event and is due to the trauma of the injection process. In some embodiments, the adverse events comprise, consist, or consist essentially of at least one of the following: swelling, bruising, tenderness, erythema and pain. In some embodiments, the adverse events include at least one of the following: edema, swelling, bruising, tenderness, erythema, pain, itching. In some embodiments, the adverse events include at least one of the following: edema, bruising, tenderness, erythema, pain, and itching.

In some embodiments, one or more of the adverse events described is avoided when applying at least one of the methods described herein. In some embodiments, the adverse events are local to the injection site. In some embodiments, the adverse events are related to the physical trauma of the inject process. In some embodiments, the adverse events include at least one adverse event selected from the group comprising, consisting, or consisting essentially of the following: erythema, edema, pain, tenderness, bruising, and itching.

The methods described herein for injecting an injectable filler can help to reduce the risk of occurrence of one or more adverse events in a subject. In some embodiments, the risk of occurrence of at least 1, 2, 3, 4, 5, 6, or 7 adverse events is reduced.

In some embodiments, the percent reduction in the risk of an adverse event occurring is substantial, for example, at least 2, 3, 4, 5, 10, 15, 20, 25, 30, 35, 40, 45, 50, 55, 60, 65, 70, 75, 80, 85, or 90 percent, including any range defined between any two of the preceding values and any range defined above any one of the preceding values. In some embodiments, the percent reduction in the risk of an adverse event occurring is near or at least 90%, e.g., 100%, such that there will be a complete or near complete likelihood of avoidance of an adverse event using the methods described. In some embodiments, the percentage reduction in the risk of one adverse event can be different from another adverse event, even if an injectable dermal treatment occurs in the same target area. In some embodiments, the likelihood that a treatment site (e.g., injection site, treatment area, etc.) will experience an adverse event is less than 50%, for example, in some embodiments, the likelihood is less than 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1%, or less.

In some embodiments, the reduction in risk of an adverse event is determined across the combination of numerous adverse events, e.g., a combination of two or more of the following adverse events: edema, swelling, bruising, tenderness, erythema, pain, and itching.

As noted herein, low injectable filler rates are associated with a reduced risk of an adverse event occurring in a subject, as shown in FIG. 1 and in the Example section below. Accordingly, in some embodiments, the mean rate of injection of an injectable filler can be maintained at less than 0.8 mL/minute. In some embodiments, the mean flow rate will be between 0.1 and 0.6 mL/minute. In some embodiments, the mean flow rate will be between 0.1 and 0.3 mL/minute. One of skill in the art will appreciate that the injection rates are not limited to the ranges above. For example, an alternative range for performing an injection can be between 0.3 and 1.2 mL/minute. Even though the risk of an adverse event can increase with increased flow rate, the flow rate need not remain constantly at a low value (e.g., less than 0.8 mL/minute) throughout the entire injection. However, additional benefits can be achieved by maintaining the flow rate at or lower than one of the injection rate values described herein throughout the entire injection. Furthermore, as noted above, in some embodiments, the reduction in the risk of an adverse event can be achieved through techniques that are independent of flow rate (such as avoiding the fanning technique or controlling the volume of injection).

In some embodiments, the mean flow rate throughout an injection can be at or lower than the flow rates discussed in the examples below and presented in Tables 1.2-1.5. For example, in some embodiments, the mean injection flow rate is less than 0.8±0.4 mL/minute. In some embodiments, the mean flow rate is 0.5±0.4 mL/min. or less. In some embodiments, the mean flow rate is 0.6±0.4 mL/min. or less. In some embodiments, the mean flow rate is less than 0.4 mL/min. In some embodiments, the flow rate is 0.5 mL/min. or less. In some embodiments, the flow rate is less than 0.65 mL/min. In some embodiments, the flow rate is 0.41 mL/min. or less.

In some embodiments, the flow rate is less than 0.63 mL/minute. In some embodiments, the flow rate is less than 0.52 mL/minute. In some embodiments, the flow rate is less than 0.5174 mL/min. In some embodiments, the flow rate is less than 0.63±0.44 mL/minute. In some embodiments, the flow rate is less than 0.19 mL/minute.

In some embodiments, the injection rate can be maintained such that the flow rate is maintained beneath a specified flow rate. In some embodiments, the flow rate of the injectable filler can be maintained below 0.8, 0.7, 0.6, 0.5, 0.4, 0.3 mL/minute or lower throughout a single injection. In some embodiments, to assist in maintaining the flow rate below a certain rate throughout the injection, a mechanical and/or electronic device can be utilized to determine the flow rate and modulate the flow rate when the flow rate approaches a specified rate. For example, in some embodiments, a spring driven mechanical device can ensure that the rate of injection is less than a specified rate (e.g., 0.4 mL/minute) throughout a single injection. In some embodiments, an electronic device can ensure that the rate of injection is less than a specified rate (e.g., 0.4 mL/minute) throughout a single injection.

In some embodiments, the injection flow rate can vary and need not stay beneath a set flow rate for the entire injection. Accordingly, in some embodiments, the flow rate will be no more than 0.8, 0.7, 0.6, 0.5, 0.4, 0.3 mL/minute for a percentage of the time of the injection, while in the remaining time, the flow rate can be higher (e.g., 1.0 mL/minute). For example, in some embodiments, the flow rate of an injectable filler can vary in a single injection such that the flow rate is no more than 0.5 mL/minute for at least 90% of the time of a single injection, while in the remaining time, the flow rate can be higher (e.g., 1.0 mL/minute). In some embodiments using more than one injection (e.g., two, three, four, five, six or more) as part of a single treatment session, the flow rate can vary in each individual injection and/or can vary between injections (e.g., a first injection can be kept constant at 0.3 mL/minute, while a second injection can be kept constant at 0.8 mL/minute). The percentage that the flow rate is kept below a certain rate can be described as a percentage of the duration of the injections throughout the entire treatment session. For example, in some embodiments, at least two injections of the injectable filler are used on a subject and the flow rate can be maintained at a rate below 0.5 mL/minute for at least 50% of the time during which the at least two injections occur.

In some embodiments, the injection rate used can depend on the type of adverse event. For some adverse events, an injection rate kept below a certain value (e.g., 0.8 mL/minute) can be sufficient to reduce the risk of the adverse events from occurring, while in other adverse events, the injection rate can be lower. In some embodiments, the injection rate can be kept at less than 0.8, 0.7, 0.6, 0.5, 0.4, 0.3 mL/minute for some injections to reduce the risk of occurrence of one type of adverse event, while for another adverse event, the injection rate can be kept higher than 0.8 mL/minute.

In some embodiments, the injection rate used can depend on the location of the target area on the subject. In some embodiments, the injection rate can depend upon the possible adverse events specific to the target area. For a target area where the subject is prone to having an adverse event, the flow rate of injectable filler can be lowered to reduce the risk of the adverse event from occurring, while the flow rate can be kept higher at the other target area. For example, for a subject who is receiving treatment in two different target areas, the first of which is sensitive to an adverse event, while the second is not, an injectable filler can be kept constantly at below 0.3 mL/minute for the entire treatment session of the first target area. However, for the second target area, the injectable filler need not be kept constantly below 0.3 mL/minute throughout the entire treatment session, but rather can vary between, e.g., 0.2 mL/minute and 0.8 mL/minute (or even higher).

One of skill in the art will appreciate that different ranges of injection rates can exist for different target areas. In some embodiments, while an injection flow rate of between 0.1 mL/minute and 1.1 mL/minute can be suitable for one target area, an injection flow rate of less than 0.3 mL/minute can be useful to reduce the risk of an adverse event for another target area.

In some embodiments, the administrator has a specific injection rate or range in mind when the injection is occurring. The administrator of the injection can have the rate of injection in mind, or can be provided with information (such as by written or oral directions) to maintain the injection rate at a specific injection rate or range. In some embodiments, the administrator will have an upper range in mind for the injection rate which the administrator will not go over. In some embodiments, the upper range is less than at least one of the following: 1.0, 0.9, 0.8, 0.7, 0.6 or 0.5 mL/minute.

In some embodiments, the rate of injection can be controlled by a device or machine controlling the flow rate, which can be either variable or constant. In some embodiments, the machine can maintain the injection flow rate within a certain flow range. For example, the machine can ensure that the flow rate of a single injection is kept between 0.01 mL/minute and 0.8 mL/minute. In some embodiments, the machine can maintain the injection flow rate below a certain rate. For example, the machine can ensure that the flow rate of a single injection is less than 0.8, such as less than 0.7, 0.6, 0.5, 0.4, 0.3, or 0.2 mL/minute.

In some embodiments, the device can be mechanical and/or include an electronic aspect. In some embodiments, the device can include a resisting member that prevents the rate of injection from increasing beyond a certain rate. In some embodiments, the device can be a programmable device capable of automated flow. In some embodiments, the device includes software that controls the flow rate of injection. In some embodiments, the software is fixed in a memory device. In some embodiments, the software controls the flow rate and/or volume injected so as to achieve any of the herein disclosed method embodiments. In some embodiments, the role of the administrator is replaced by the software in the device.

In some embodiments, the treatment employs any injectable filler. In some embodiments, a hyaluronic acid (“HA”) injectable filler is used. In some embodiments, the HA contains about 100,000 gel particles/ml filler. In some embodiments, the treatment does not include HA. In some embodiments, the filler allows for expansion of the tissue while also being capable of being absorbed into the skin or degraded.

In some embodiments, the hyaluronic acid is generated by a Streptococcus species of bacteria. In some embodiments, the hyaluronic acid is stabilized, e.g., non-animal stabilized. In some embodiments, the hyaluronic acid is chemically crosslinked with BDDE (1,4 butanediol diglycidyl ether), stabilized (e.g., NASHA), and suspended in phosphate buffered saline at a pH of 7 and a concentration of 20 mg/ml. In some embodiments, the hyaluronic acid is free of animal protein. For example, in an embodiment, the hyaluronic acid is a gel generated by a Streptococcus species of bacteria, chemically cross-linked with BDDE, stabilized, and suspended in saline at pH 7 (e.g., as in RESTYLANE® injectable filler, RESTYLANE TOUCH™ injectable filler, RESTYLANE FINE LINES™injectable filler, RESTYLANE VITAL™ injectable filler, and RESTYLANE LIPP™ injectable filler). Such embodiments can be in a concentration of 20 mg/ml, phosphate buffered at pH 7, and/or free of animal protein. In some embodiments, the hyaluronic acid is one that is suitable for injection into a dermal location where it acts to stimulate collagen synthesis.

In some embodiments, the hyaluronic acid is in the form of gel particles. In some embodiments, the hyaluronic acid is in the form of gel particles having sizes in the range of about 940 microns to about 1090 microns. In some embodiments, the largest fraction of gel particles size is between 940 microns and 1090 microns (e.g., as in PERLANE® injectable filler). In some embodiments, the hyaluronic acid gel particles have a particle size that is less than 1200 microns. In some embodiments, the hyaluronic acid gel particles have a particle size that is about 400 microns. In some embodiments, the hyaluronic acid gel particles have a particle size that is less than 400 microns. In some embodiments, the hyaluronic acid gel particles have a particle size that is more than 400 microns. In some embodiments, the hyaluronic acid gel particles have a particle size that is in the range of about 400 to about 1200 microns.

The concentration of hyaluronic acid gel particles in the injectable filler can vary over a broad range, e.g., about 500-200,000 particles per mL, such as about 500-5000 particles per ml, about 5,000-50,000 particles per ml, about 50,000-150,000 particles per ml, or about 150,000-200,000 particles per ml. For example, in some embodiments, the injectable filler comprises about 200,000 hyaluronic acid gel particles per ml (e.g., as in RESTYLANE FINE LINES™ injectable filler and RESTYLANE TOUCH™ injectable filler). In some embodiments, the injectable filler comprises about 100,000 hyaluronic acid gel particles per ml (e.g., as in RESTYLANE® injectable filler). In some embodiments, the injectable filler comprises about 10,000 hyaluronic acid gel particles per ml (e.g., as in PERLANE® injectable filler). In some embodiments, the injectable filler comprises about 1,000 hyaluronic acid gel particles per ml (e.g., as in RESTYLANE SUBQ™ injectable filler). The package inserts for RESTYLANE® injectable filler and PERLANE® injectable filler are hereby incorporated by reference in their entireties, and particularly for the purpose of describing those brands of injectable filler products.

In some embodiments, the hyaluronic acid composition comprises a cross-linked biocompatible polysaccharide gel composition, which is obtainable by cross-linking a cross-linkable polysaccharide with a polyfunctional cross-linking agent in two steps, the first cross-linking step can be terminated before gelation occurs by a sterical hindrance of the cross-linking reaction. The second cross-linking step can be initiated by reintroducing sterically unhindered conditions for the cross-linking reaction. This reaction can continue up to a viscoelastic gel, wherein the gel composition exhibits retained biocompatibility, viscoelasticity and does not swell substantially when placed in contact with water. In some embodiments, the stabilized hyaluronic acid composition is that disclosed in U.S. Pat. No. 5,827,937, hereby incorporated by reference in its entirety and particularly for the purpose of describing hyaluronic acid compositions and methods of making them.

In some embodiments, the stabilized hyaluronic acid can be prepared as described in U.S. Pat. No. 5,827,937. In some embodiments, this process can include the following steps: forming an aqueous solution of a water soluble, cross-linkable polysaccharide; initiating a cross-linking of said polysaccharide in the presence of a polyfunctional cross-linking agent therefore; sterically hindering the cross-linking reaction from being terminated before gelation occurs (thereby obtaining an activated poly-saccharide); and reintroducing sterically unhindered conditions for said activated polysaccharide so as to continue the cross-linking thereof. In some embodiments, the process involves a cross-linking of a water-soluble, cross-linkable polysaccharide in at least two steps or stages, where the cross-linking reaction is discontinued before the galation is initiated. The discontinuance can be accomplished by sterically hindering the cross-linking reaction. The cross-linking reaction can then be continued in a second step by reintroducing sterically unhindered conditions. Any known cross-linking agent can be used, if it is useful in connection with polysaccharides, consideration being taken to ensure that the biocompatibility prerequisites are fulfilled. Preferably, however, the cross-linking agent is selected from the group consisting of aldehydes, epoxides, polyaziridyl compounds, glycidyl ethers and dividylsulfones. Of these glycidyl ethers represent an especially preferred group, of which 1,4-butandiol digylcidylether can be referred to as a preferred example. The initial cross-linking reaction in the presence of a polyfunctional cross-linking agent can be performed at varying pH values, primarily depending on whether ether or ester reactions should be promoted. Preferably this means that said cross-linking reaction is performed at an alkaline pH, especially above pH 9, e.g. in the range of pH 9-12, when promoting ether formations. When promoting ester formations said cross-linking reaction is preferably performed at an acidic pH, especially at pH 2-6. In some embodiments, the activation of the polymer can occur under alkaline conditions and as follows: 10 g of hyaluronic acid from Streptococcus can be dissolved in 100 ml of 1% NaOH pH>9. Cross-linking agent in the form of 1,4-butandiol diglycidylether can be added to a concentration of 0.2%. The solution can be incubated at 40 degrees Celsius for 4 hours. In some embodiments, the activation of the polymer can occur under acidic conditions and as follows: similar as above, but at an acidic pH of about 2-6 by the addition of 1% of acetic acid to the solution instead of NaOH.

In some embodiments, the filler is a biosynthesized, non-animal hyaluronic acid (e.g., as in JUVEDERM® injectable filler). In some embodiments, the filler is in the form of an injectable gel. In some embodiments, the injectable gel can include rough particles, while in other embodiments, the gel is generally of smooth consistency.

In some embodiments, the filler is comprised of polymethylmethacrylate (PMMA) microspheres suspended in bovine collagen (e.g., ARTEFILL™ injectable filler). In some embodiments, the microspheres that are suspended in the bovine collagen can become part of the patient's own skin. In some embodiments these microspheres can have a defined size of 30 to 50 microns in diameter. In some embodiments, the polymethylmethacrylate microspheres comprises approximately 20% of the total volume of the filler composition. In some embodiments, the filler is in the form of an injectable gel. In some embodiments, portions of the fillers can resist being taken up by scavenger cells (macrophages) and can avoid being degraded by enzymes.

In some embodiments, the filler is comprised of cross-linked hyaluronic acid from biofermentation (e.g., from ARTEMIS™ injectable filler). In some embodiments, the filler will have little or no allergenic effects.

In some embodiments, the filler is comprised of poly-L-lactic acid (e.g., as in SCULPTRA™ injectable filler). In some embodiments, the filler will be used for restoration and/or correction of the signs of facial fat loss, or lipoatrophy, by replacing lost volume. In some embodiments, the filler will be a biocompatible and/or biodegradable material.

In some embodiments, the filler is comprised of microspheres of at least calcium and phosphate ions (e.g., as in RADIESSE®, injectable filler). In some embodiments, the filler is comprised of calcium based microspheres suspended in a water-based gel. In some embodiments, the microspheres can be calcium hydroxylapatite (CaHA).

Although various injectable fillers have been described above, one of skill in the art will appreciate that various injectable fillers can be used alone or in combination to treat specific target areas. In some embodiments, using the methods provided above, any combination of the dermal fillers can be used while achieving a reduction in the rate of an adverse event in a subject.

In some embodiments, any of the injectable fillers described herein, as well as any of the volumetric and/or dermal fillers described herein, can be used in one or more of the herein disclosed methods.

In some embodiments, any injectable filler whose application results in a same, similar, or overlapping set of adverse events as described herein can also benefit from one or more of the presently disclosed methods. Thus, in some embodiments, any injectable filler that when used, can result in one or more of the following adverse events: itching, tenderness, bruising, redness, pain, redness, and swelling, can benefit by one of more of the herein disclosed events. Exemplary injectable fillers include those disclosed herein. As is appreciated by one of skill in the art, the various side effects of the various injectable fillers can be reviewed in various publications, including the labels accompanying the various injectable fillers. Exemplary labels, which include such adverse events, include the labels for the following: RESTYLANE® injectable filler, PERLANE® injectable filler, JUVEDERM® 30 injectable filler, ELEVESS™, injectable filler, HYLAFORM® injectable filler, and RADIESSE®, injectable filler, the entirety of each of which is incorporated by reference. As noted in these labels, the adverse events that occur are very similar, even if the compounds are not, for example, RESTYLANE® injectable filler can result in the following adverse events: bruising, redness, swelling, pain, tenderness, itching, pimples, sore throat, and runny nose; PERLANE® injectable filler can result in the following adverse events: bruising, redness, swelling, pain, tenderness, itching, and pimples; JUVEDERM® 30 injectable filler can result in the following adverse events: firmness, redness, swelling, pain/tenderness, lumps/bumps, discoloration, and itching; ELEVESS™ injectable filler can result in the following adverse events: bruising, redness, swelling, pain, tenderness, itching, and nodule formation; HYLAFORM® injectable filler can result in the following adverse events: erythema, bruising, swelling, pain, pruritus, and desquamation; and RADIESSE® injectable filler can result in the following adverse events: ecchymosis, edema, erythema, granuloma, nodule, pain, pruritus, contour irregularities, numbness, dryness, peeling, burning sensation, whiteheads, and rash. Given the high degree of overlap and similarity in these adverse events between these various injectable fillers, it appears evident that the herein disclosed methods will function across a large variety of injectable fillers. In particular situations, routine experimentation informed by the guidance provided herein can be used by those skilled in the art to identify suitable injection conditions. Furthermore, in the study conducted as generally set forth in the examples below, there was a strong correlation found between the type of injection technique, volume of delivery, and rate of delivery, as opposed to the actual agent delivered. Therefore, one of skill in the art will appreciate that address one or more of these aspects can provide the benefit of reducing the risk of an adverse event occurring, regardless of the type of filler that is used.

In some embodiments, some of the locations that can be addressed by the systems and methods disclosed herein include: oral commissure, marionette lines, mandibular hollow, raise jowl, frowning mouth, pout lower lip, later expression lines, mental crease, chin dimpling, zygomatic hollow, nasolabial folds, tear trough, and brow lift.

In some embodiments, the objective of the treatment can be to achieve a desired cosmetic result at the area of treatment. In some embodiments, defects (e.g., areas or locations that appear to lack volume or are “under volume”) can be fully corrected during a treatment session. The amount of correction can be ascertained by visual assessment of appearance of the defect. In some embodiments, the amount of correction can be determined with the aid of, for example, a Wrinkle Severity Rating Scale (WSRS), such as that shown in Table 0.1.

TABLE 0.1
Wrinkle Severity Rating Scale

| Score | Description |
|---|---|
| 5 | Extreme: Extremely deep and long folds; detrimental to facial |
| | appearance. 2-4 mm visible v-shaped fold when stretched. |
| | Unlikely to have satisfactory correction with injectable |
| | implant alone. |
| 4 | Severe: Very long and deep folds; prominent facial feature. Less |
| | than 2 mm visible fold when stretched. Significant improvement is |
| | expected from injectable implant |
| 3 | Moderate: Moderately deep folds; clear facial feature visible at |
| | normal appearance but not when stretched. Excellent correction is |
| | expected from injectable implant. |
| 2 | Mild: Shallow but visible fold with a slight indentation; minor |
| | facial feature. Implant is expected to produce a slight improvement |
| | in appearance. |
| 1 | Absent: no visible fold; continuous skin line |

For example, a severity rating of 1 on the severity scale provided above can indicate full correction of a defect. In various embodiments, overcorrection can be undesirable. In various embodiments, treatment and/or re-treatment can correct defects from about 90% to about 100%. Preferably, a maximum of about 100% correction should be administered, without overcorrection, at each treatment.

In some embodiments, the injection site can be massaged to conform to the contour of the surrounding tissues.

In some embodiments, the amount of filler composition administered at each session for any target area can be in the range of from about 0.01 cc to about 3 cc, for example 0.01-0.05, 0.05-0.1, 0.1-0.15, 0.15-0.2, 0.2-0.3, 0.3-0.4, 0.4-0.5, 0.5-0.6, 0.6-0.7, 0.7-0.8, 0.8-0.9, 0.9-1, 1-1.2, 1.2-1.4, 1.4-1.6, 1.6-1.8, 1.8-2, 2-2.2, 2.2-2.4, 2.4-2.6, 2.6-2.8, 2.8-3 cc. In some embodiments, each treatment site can be treated with, for example, a maximum dosage of about 1-2, 2-3, 3-4, 4-5 cc per treatment session. If the treated area is swollen directly after the injection, melting ice can be applied on the site for a short period. The subject can be evaluated post treatment, which is described in more detail below. In some embodiments, photographs can be taken prior to each treatment. In some embodiments, the photography can be done in accordance with, for example, the standard Canfield system.

In some embodiments, the methods disclosed provide a favorable change of at least one score in a Wrinkle Severity Rating Scale. In some embodiments, the methods disclosed provide a favorable change of at least two scores in a Wrinkle Severity Rating Scale.

In various embodiments, aesthetic improvement can also be evaluated for global aesthetic improvement, e.g. improvement from pre-treatment appearance. In some embodiments, the following exemplary categorical scale (in Table 0.2) can be used to measure global aesthetic improvement:

TABLE 0.2
Global Aesthetic Improvement Scale

| Rating | Definition |
|---|---|
| Very Much | Optimal cosmetic result for the implant in this subject. |
| Improved | |
| Much Improved | Marked improvement in appearance from the initial |
| | condition, but not completely optimal for this subject. |
| Improved | Obvious improvement in appearance from the initial |
| | condition. |
| No Change | The appearance is essentially the same as baseline. |
| Worse | The appearance is worse than the original condition. |

In some embodiments, point values, scores, or grades can be assigned to the above groups. In some embodiments, the point values, scores, or grades are as follows: 0—Worse; 1—No Change; 2—Improved; 3—Much Improved; and 4—Very Much Improved.

In some embodiments, evaluation can be made in view of the overall cosmetic result for each area of interest. The ratings can be correlated with the actions that would generally be considered in the normal course of practice. Review of a pre-treatment archival photograph (obtained prior to initial treatment) at each follow-up session can aid in the assessment. In some embodiments, the methods disclosed provide a favorable change of at least one step in a Global Aesthetic Improvement Scale. In some embodiments, the methods disclosed provide a favorable change of at least two steps in a Global Aesthetic Improvement Scale. In some embodiments, the methods disclosed provide a favorable change of at least three steps in a Global Aesthetic Improvement Scale.

In some embodiments, the assessment is performed at, for example, about two weeks after treatments and at each subsequent visit.

In some embodiments, the treatment site can be cleaned with a suitable antiseptic solution. The injectable filler can be administered using, for example, a thin gauge needle by injecting the material into, for example, the deep dermis and/or the surface layer of subcutis. In some embodiments, if the injectable filler is injected too deep or intramuscularly, the duration of the implant can be shorter because of a higher filler turnover rate. In some embodiments, too superficial an injection can give blanching effects and bumps on the treatment site. Before injecting, the air can be removed from the syringe up to the point where a droplet is visible on top of the needle.

The injection technique with regard to the depth of injection and the administered quantity can vary. A variety of injection techniques are known in the art and can be used in conjunction with the embodiments described herein. In various embodiments, the linear threading technique can be used to carefully lift up a wrinkle or fold. In other embodiments, a series of punctual injections or a combination of the two techniques can be used. In some embodiments, the eye of the needle preferably faces upwards during injection. In some embodiments, the contour of the needle can preferably be visible. In some embodiments, the injectable filler is injected while pulling the needle slowly backwards. Injection can stop just before the needle is pulled out from the skin to prevent material from leaking out from the injection site.

In some embodiments, concomitant medications or other treatments can be utilized when medically necessary. Concomitant medications, can include, for example, over-the-counter (OTC) medications, and procedures such as, for example, surgery/biopsy or diagnostic evaluations.

In some embodiments, methods and systems disclosed herein provide for ease of training or instruction for improving the efficacy of administering an injectable filler. As is appreciated by one of skill in the art, to some extent, the application of injectable fillers in the cosmetics industry can be characterized as more of an “art” than a science. In other words, training people in this process can often be a trial and error experience rather than involving a clear set of instructions and signposts to follow. The present discovery represents a scientific realization, that can readily be passed to others for appropriate execution; thereby removing some of the “art” aspect of the application of the injectable fillers.

Thus, in some embodiments, provided herein are systems and methods that can be, relatively speaking, readily and/or clearly taught. In some embodiments, this involves teaching others how to perform the treatment methods described herein. In some embodiments, the low adverse event methods lend themselves to ready communication to others and discussion of how and why the technique works. Additionally, in some embodiments, the techniques can be readily applied by numerous and different people with different backgrounds. That is, in some embodiments, the teaching of the above methods provide for increased reproducibility of the results described herein with the relevant products. In some of the embodiments, providing users with the knowledge of these methods provides quality control for improving the efficacy of a filler composition. Thus, in some embodiments, a method for teaching a technique that is especially amenable to teaching (and/or the other aspects noted above) is provided. In some embodiments, the teaching of the method itself also provides the above noted advantages of providing users with a basic technique in common, reproducibility and predictability of results, and allowing a broader range of people to apply the filler. Of course, the application of the technique itself can have the specific advantages noted herein as well.

In one embodiment, a method of teaching is provided that reduces the likelihood of an occurrence of an adverse event. The method involves providing information to one or more individuals, wherein the information comprises a technique to reduce a likelihood of an adverse event occurring in a subject matter due to an application of an injectable filler to the subject. In some embodiments, the information comprises directions regarding an injectable filler and that one should avoid applying more than 5 mL of injectable filler volume in a single treatment session. In some embodiments, the information directs one to avoid a flow rate of injectable filler of greater than 0.8 mL/minute.

In some embodiments, a method of teaching a technique to reduce a likelihood of an occurrence of an adverse event in a subject is provided. The method can include providing information to one or more individuals. The information can include a technique to reduce a likelihood of an adverse event occurring in a subject due to an application of an injectable filler to the subject. The information can comprise directions to avoid the application of greater than 5 mL of injectable filler volume in a single treatment session. The information can also include directions to avoid a flow rate of an injectable filler of greater than 0.8 mL/minute.

In some embodiments, information can be provided to one or more individuals in a classroom, via a pamphlet, telephone transmission, video transmission or via a web-based form of communication.

In some embodiments, a method of distributing an injectable filler is provided. The method comprises providing an injectable filler to an injectable filler administrator and providing a set of instructions regarding how to apply the injectable filler to the injectable filler administrator. The set of instructions instruct the administrator to inject the injectable filler at a rate of no more than 0.8 mL/min. The set of instructions can be provided orally (such as by word of mouth) or visually (such as by printed paper).

In some embodiments, a method of doing business is provided. In some embodiments, an advertisement is provided and/or distributed. The advertisement can advertise the application of dermal filler in a manner that results in reduced, fewer, or no side effects or adverse events (or similar such statement). Following this, one applies one or more of the herein noted techniques (which reduce the likelihood of adverse events) to a subject. In some embodiments, the subject sees the advertisement. In some embodiments, money is collected for the technique performed on the subject. Because of this, greater peace of mind can be had by the subject receiving the dermal filler, as well as superior results.

In some embodiments, a method of selling an injectable filler to an administrator is provided. The method can comprise advertising a rate of adverse events to an administrator. The rate of adverse events is a rate that occurs in a population of subjects that has received an injectable filler at an injection flow rate of no more than 0.6 mL per minute (e.g., 0.3 mL/minute). The method can further comprise selling the injectable filler to the administrator. The injectable filler is distributed with a set of instructions that instruct an administrator to inject the injectable filler at a flow rate of no more than 0.6 ml per minute (e.g., 0.3 mL/minute).

In some embodiments, a method of setting a price on an injectable filler is provided. The method can comprise pricing an injectable filler at a first price based on a first rate of adverse events and advertising a likelihood of an adverse event occurring from an administration of the injectable filler to a subject. The likelihood of an adverse event occurring is determined from an administration of the injectable filler at a flow rate of less than 0.8 mL/minute (e.g., 0.3 mL/minute). The method can further comprise repricing the injectable filler at a second price. At least a part of a difference in the first price and the second price is due to a value perceived by an injectable filler administrator in the likelihood of the adverse event occurring.

In some embodiments, a method of distributing an injectable filler is provided. The method can comprise providing an injectable filler to an injectable filler administrator and providing a set of instructions regarding how to apply the injectable filler to the injectable filler administrator. The instructions instruct the administrator to inject the injectable filler at a mean flow rate of no more than 0.8 mL/minute (e.g., 0.7, 0.6, 0.5, etc.).

In some embodiments, a kit for decreasing the risk of an adverse event associated with the administration of an injectable filler is provided. The kit can include an injectable filler, a syringe, a needle, and instructions or guidance for performing part, some, or all of the above methods. In some embodiments, the injectable filler is RESTYLANE® or PERLANE® injectable filler. The instructions can be provided on a variety of formats, such as electronic (data file, DVD, downloadable, etc) or pamphlets. The syringe can be a 4 mL or smaller syringe (e.g., 2 mL). In some embodiments, the syringe is prefilled with the injectable filler. In some embodiments, the kit comprises a first plurality of syringes, wherein each of the first plurality of syringes has a first volume. In some embodiments, the first volume is between 1 and 5 ml, e.g., 1, 2, 3, 4, or 5. In some embodiments, the kit includes gloves. In some embodiments, the kit includes sterilizing material. In some embodiments the kit includes a cloth or other absorbent material.

In some embodiments, the kit includes software for assisting in capturing images of the subject's face. In some embodiments, the software compares two facial images of the subject and determines where one should inject the injectable filler by identifying the areas that appear to lack volume or appear to have lost volume. In some embodiments, the software compares two facial images of the subject and assesses improvement or worsening of wrinkle severity.

In some embodiments, a kit for reducing a risk that an adverse event will occur in a subject is provided. The kit can comprise an injectable filler, a set of instructions for administering the injectable filler (the instructions can provide that the injectable filler should be injected at a flow rate of no greater than 0.8 mL/minute, such as 0.3 mL/minute), and an injection device for applying the injectable filler to a subject. In some embodiments, the injectable filler comprises at least one injectable filler selected from the group consisting of: non-animal stabilized hyaluronic acid, biosynthesized non-animal hyaluronic acid, polymethylmethacrylate microspheres suspended in bovine collagen, cross-linked hyaluronic acid from biofermentation, poly-L-lactic acid, and microspheres of at least calcium and phosphate ions.

In some embodiments, a training kit is provided. The kit can include instructions or guidance for performing parts of or all of some or all of the above techniques. The instructions can be provided on a variety of formats, such as electronic (data file, DVD, downloadable, etc) or pamphlets. The instructions can generally provide one with any of the steps outlined herein. For example, the instructions can include information regarding how much to inject, how to touch-up after an injection, techniques for maintaining a relatively slow rate of injection, the amount of time it can take for a procedure and for patient recovery after the procedure, the amount of pain that occurs during the procedure, advantages of the present methods over other methods, the results that can be expected, and how the injections should be made in particular situations. In some embodiments, the training kit includes before and after depictions of subjects that have received the treatment. In some embodiments, the kit includes depictions from injectable filler applications that employed one or more of the herein presented methods and depictions of injectable filler applications that did not employ the herein presented methods.

As will be appreciated by one of skill in the art, in some embodiments, the training kit not only provides training for the applicator of the technique, but can also provide additional information to help the applicator sell the technique to potential clients. In some embodiments, the kit includes information to help the applicator order additional injectable filler.

A number of devices can be applied to assist in maintaining the flow rate of an injectable filler at the desired rate, and thereby reduce the risk of an adverse event from occurring. In some embodiments, the device is a syringe and/or needle. In some embodiments, the dermal filler is applied via a syringe and/or needle that facilitates the methods of applying the dermal filler (e.g., slow application of the dermal filler) as described above. In some embodiments, there is a flow restrictor or a very narrow gauge needle is used (e.g., 30 to 32 cc.) to slow the application of the dermal filler. In some embodiments, the syringe and/or needle facilitates multiple puncture and/or linear/serial threading and/or lower volumes. In some embodiments, the dermal filler is contained within such a syringe as part of a kit. In some embodiments, the kit includes instructions describing some or all of the techniques and/or aspects described herein.

In some embodiments, a method for reducing a risk of an adverse event occurring from the administration of an injectable filler is provided. The method comprises providing instructions that the injectable filler should be applied at an injection rate of 0.8 mL/minute or slower. The instructions are on a computer readable medium. In some embodiments, the instructions are displayed on a website, and the website is accessible to an injectable filler administrator. In some embodiments, the instructions are recorded verbal instructions, and wherein the instructions are played over a phone line or video connection to an injectable filler administrator.

In some embodiments, a method of reducing a rate of adverse events associated with an injectable filler in a population of subjects is provided. The method comprises informing one or more injectable filler administrators that the injectable filler is to be injected at a mean flow rate of no more than 0.8 ml/minute (e.g., 0.7, 0.6, 0.5, etc.) The population of subjects can have the injectable filler injected by the one or more injectable filler administrators. In some embodiments, the flow rate is no more than 0.3 mL/minute.

In some embodiments, a method of treating a subject with an injectable filler is provided. The method can comprise identifying a subject that is to receive an injectable filler. In some embodiments, the subject desires to reduce a likelihood of any adverse events occurring from applying the injectable filler. The method further comprises applying the injectable filler to the subject and the administrator selects or slows down a flow rate of the injection of the injectable filler such that a mean flow rate of the injectable filler is less than 0.5 mL/minute for all injections of the injectable filler in a treatment session.

In some embodiments, a method of applying an injectable filler is provided. The method comprises informing a subject to receive an injectable filler of two options. The first option comprises a shorter treatment session and a higher risk of an adverse event. The second option comprises a longer treatment session and a lower risk of an adverse event. One then further receives the selected choice of either the first or the second option from the subject and then applies the injectable filler to the subject. If the subject selects the first option, the injectable filler is injected at a mean rate flow that is above 0.6 mL/min., and wherein if the subject selects the second option, the injectable filler is injected at a mean flow rate that is below 0.6 mL/min.

In some embodiments, a method of applying an injectable filler to a subject is provided. The method comprises injecting a subject with an injectable filler at a first rate, observing an adverse event at an injection site on the subject, and injecting the subject with the injectable filler at a second rate. The second rate is slower than the first rate, and the second rate is less than 0.8 mL/min.

In some embodiments, a method of applying an injectable filler to a subject is provided. The method comprises injecting a subject with an injectable filler at a flow rate that is less than 0.8 mL/min, and informing the subject of the risk that they will experience an adverse event from the injection of the injectable filler. The risk is no more than a risk associated with an injection of a dermal filler at a flow rate of no more than 0.8 mL/minute. In some embodiments, the flow rate (and corresponding risk for the flow rate) is no more than 0.3 mL/min.

As noted above, in some embodiments, one can employ a relatively slow flow rate during the injection of an injectable filler and achieve superior and unexpected benefits in a reduction in the risk of an adverse event occurring.

In some embodiments, using the appropriate flow rate allows one to avoid various types of adverse events. In some embodiments, the risk that the injection will result in the adverse event of swelling can be reduced. In some embodiments, the risk that the injection will result in the adverse event of bruising can be reduced. In some embodiments, the risk that the injection will result in the adverse event of tenderness can be reduced. In some embodiments, the risk that the injection will result in the adverse event of erythema can be reduced. In some embodiments, the risk that the injection will result in the adverse event of pain can be reduced. In some embodiments, the risk of at least one of the above adverse event is reduced. In some embodiments, the risks of at least two of the above adverse events are reduced. In some embodiments, the risks of at least three of the above adverse events are reduced. In some embodiments, the risks of at four of the above adverse events are reduced. In some embodiments, the risks of all five of the above adverse events are reduced. In some embodiments, the risk that any adverse event will occur is generally reduced.

In some embodiments, the severity of any one or more of the above is reduced. In some embodiments, the severity is reduced to the point where the subject does not realize and/or mind that there is an adverse event. In some embodiments, the magnitude of the adverse event is reduced by at least some amount, for example, by at least 5, 10, 15, 20, 25, 30, 40, 50, 60, 70, 80, 90, 95, 98, 99 percent or more, including any range greater than any of the preceding values.

In some embodiments, at least one of the above adverse events is completely absent from the subject.

In some embodiments, the risk of at least one of the above adverse events is minimized or reduced. In some embodiments, the method results in the minimization of the risk of an adverse event, including at least one of the following: swelling, bruising, tenderness, erythema and pain.

In some embodiments, the odds of an adverse event occurring are reduced. In some embodiments, even though the injectable filler is administered to a single person, the likelihood that the person will experience one or more of the adverse events can be expressed as an odds. In some embodiments, the odds of an adverse event are reduced in a manner consistent with the odds presented in FIG. 1. In some embodiments, the injectable filler is administered at a flow rate to achieve an odds of less than 1, for example, less than 0.9, 0.8, 0.7, 0.6, 0.5, 0.4, 0.3, 0.2, 0.1 or lower.

In some embodiments, the method employs a flow rate of 0.8 mL/min. and results in an odd of an adverse event of 0.275. In some embodiments, the method employs a flow rate of 0.3 mL/min. and results in an odd of an adverse event of 0.15. In some embodiments, the method employs a flow rate of 0.2 mL/min. and results in an odd of an adverse event of 0.1. In some embodiments, the graph in FIG. 1 is used or can describe the odds for a given flow rate.

In some embodiments, at least 0.1 mL of injectable filler is administered in a treatment session, for example, in some embodiments, about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.21, 3.3, 3.4, 3.5, 3.6, 3.7, 3.8, 3.9, 4, 4.1, 4.2, 4.3, 4.4, 4.5, 4.6, 4.7, 4.8, 4.9, 5, or more milliliters of the injectable filler are applied during the treatment session and still result in a reduction in the risk of an adverse event.

In some embodiments, not more than 6 mL of the injectable filler are injected into the subject in a single treatment session, thereby providing a further reduction in the rate or likelihood of an adverse event. In some embodiments, less than 3 mL is applied to a subject in any one treatment session. In some embodiments, this can be less than any of the following values: 3, 2.9, 2.8, 2.7, 2.6, 2.5, 2.4, 2.3, 2.2, 2.1, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1 mL per treatment session of the nasolabial folds.

In some embodiments, the volume of injectable filler applied at any one treatment site is relatively low and thereby reduces the risk that an adverse event will occur. In some embodiments, the volume is less than 1.1 mL at any one treatment site, for example, 1, 0.9, 0.8, 0.7, 0.6, 0.5, 0.4, 0.3, 0.2, 0.1 or less mL of the injectable filler. In some embodiments, the volume of injectable filler applied at a single treatment site is less than 1.04 mL. In some embodiments, the volume is 0.8 mL or less.

In some embodiments, a risk of an adverse event is reduced, even though the subject's WSRS is reduced by at least 1 level, for example, 1, 2, 3, or more levels. In some embodiments, the risk of an adverse event is reduced, event though the subject's GAIS has increase to at least 2, 3, or 4 on the GAIS.

In some embodiments, a risk of an adverse event is reduced, even though the subject has received a full correction.

In some embodiments, the method provides for a reduction in the risk of an adverse event, regardless of the type of injection technique that is employed. In other embodiments, the method provides for a reduction in the risk of an adverse event from occurring by avoiding certain types of injection techniques but does not require one to monitor or control the flow rate. As noted above and explained in the examples below, in some embodiments, one can reduce the risk of an adverse event from occurring by avoiding various injection techniques, such as the fanning technique. In some embodiments, the injection technique is selected from the group consisting essentially of at least one of the following: serial puncture, linear threading, and serial threading injections. In some embodiments, the injection technique is selected from the group consisting essentially of at least one of the following: serial puncture, linear threading, serial threading and cross-hatching injections. In some embodiments, the injection technique reduces or avoids dissection of the sub-epidermal plane with lateral movement of the needle.

In some embodiments, the method provides further advantages when the slower flow rate is used with techniques that also further reduce the risk of an adverse event, such as serial and threading techniques. Thus, further advantages can be obtained by avoiding the use of the fanning injection technique.

In other embodiments, the technique of fanning can be employed.

In some embodiments, the method comprises the step of identifying a subject to receive the injectable filer by a method described herein. In some embodiments, the subject can be identified as one that would like to minimize any defects or adverse events following the application of the dermal filler. In some embodiments, the subject can be identified as one on which the relevant adverse events could be particularly obvious. In some embodiments, the subject can be identified by being a subject that is receiving an injectable filler, where the instruction or label accompanying the injectable filler instructs the administrator to inject the injectable filler at a flow rate of less than 0.8 mL/min, e.g., less than 0.3 mL/minute.

In some embodiments, one can determine that a subject has been identified by the scheduling of relatively longer treatment session, such as a treatment session that is at least long enough so as to allow for the application of the dermal filler at a slower injection rate (e.g., less than 0.8 mL/minute, such as 0.7, 0.6, 0.5, 0.4, 0.3, 0.2 mL/minute or less).

In some embodiments, the subject is identified as one that can benefit from a slower injection rate as part of a general approach on the part of the administrator to generally reduce the risk of adverse events in the administrator's subjects.

In some embodiments, the subject is identified by asking the subject how sensitive they are to one or more of any of the adverse events and then injecting at the appropriate rate.

In some embodiments, the subject is selected as one that should receive the maximum benefit available from the injectable filler, and thus, the herein disclosed method is then appropriate for the subject.

In some embodiments, the subject is selected based upon a perceived risk on the part of the administrator that the subject can suffer from an adverse event and that the relatively slower injection rate can reduce the risk of an adverse event.

In some embodiments, the subject is identified by giving the subject the choice of either a shorter treatment session, with risk of adverse events, or a longer treatment session, with a lower risk of adverse events. In some embodiments, the subject notes a preference regarding the risk of adverse events and thereby allows the applicator to identify the subject.

In some embodiments, at least one of the herein disclosed methods is used on at least one target area on a subject. In some embodiments, as the method can reduce the risk of an adverse event at the target area, the method can be applied to a specific target area that is susceptible to adverse events. Thus, in some embodiments, it can be advantageous to identify a specific target area on the subject that is to receive at least one of the methods disclosed herein. In some embodiments, at least one of the herein disclosed methods is applied to a location on a subject that is especially sensitive or prone to one or more of the adverse events. In some embodiments, the location is especially sensitive to one or more of: swelling, bruising, tenderness, erythema and pain.

In some embodiments, the location is one that the subject is especially concerned or sensitive about.

In some embodiments, the location is one that is visible and/or prominent to viewers of the subject's face. Thus, prominent areas can be targeted for the presently disclosed methods, while less prominent areas do not need to be included.

In some embodiments, the location is one that the administrator is concerned about the occurrence of adverse events in. The administrator can identify this location as important to the subject, susceptible to the adverse events, or as a highly prominent area on the subject.

In some embodiments, the location to receive the disclosed method is selected by the type of location, the depth of the location and/or the impact of an adverse event on the location.

As will be appreciated by one of skill in the art, each of the above locations is a location where the injection method can be especially useful and can first be identified by an administrator as an area that would benefit from the method. Following this identification, the method can then be applied to that location.

As will be appreciated by one of skill in the art, the present method can be performed with various types of injections, at various locations, and at various depths in the subject's skin.

In some embodiments, the location is selected from the group consisting of at least one of: oral commissures, marionette lines, mandibular hollows, raise jowls, frowning mouth, pouty lower lip, lateral expression lines, mental creases, chin dimplings, zygomatic hollows, nasolabial folds, tear troughs, malar area or prominence, and brow lifts. In some embodiments, the location is an oral commissure or a nasolabial fold.

In some embodiments, the depth of the location of injection can be the mid-dermis, the deep dermis, the superficial layer of the subcutis, or any combination thereof or any space therebetween. In some embodiments, the location of the injection can be at location in the skin that the injectable filler can be applied to.

### EXAMPLES

An extensive study was conducted on 283 subjects as generally described below to determine the relationship between dermal filler injection techniques and local adverse events. The study examined the impact of injection technique on the occurrence of local adverse events due to the implantation of hyaluronic acid fillers. “Local adverse events” was defined as any adverse experience to the patient including pain, tenderness, redness, ecchymosis, swelling, itching, mass formation (nodule, cyst, or abscess) or other events at the site of injection. The study was performed on 283 subjects with moderate-to-severe wrinkles (including nasolabial folds and oral commissures) undergoing treatment with injectable dermal fillers. 142 subjects were injected with nonanimal-stabilized hyaluronic acid gel particles 400 μm in size (NASHA-small, RESTYLANE®), while 141 subjects were injected with nonanimal-stabilized hyaluronic acid gel particles 1,000 μm in size (NASHA-large, PERLANE®). There were no limitations on injection techniques. The subjects were assessed at 72 hours and 2, 6, 12, and 24 weeks for local adverse events.

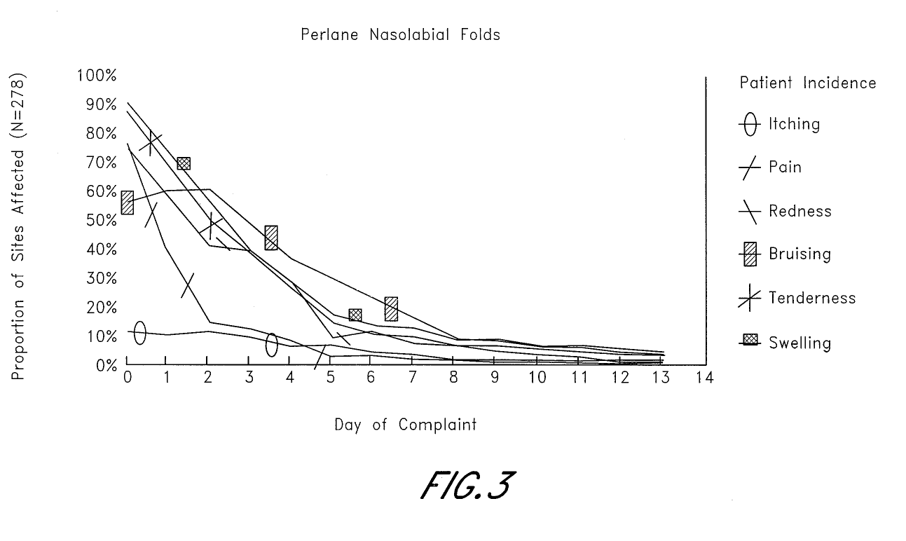

FIG. 3 is a graph depicting the sum of the adverse events experienced by subjects to the hyaluronic acid dermal fillers. The data for both the RESTYLANE® and PERLANE® dermal fillers was comparable. 278 of the 283 subjects disclosed experiencing a number of adverse events, including itching, pain, redness, bruising, tenderness and swelling. One or more of these adverse events lasted for at least thirteen days.

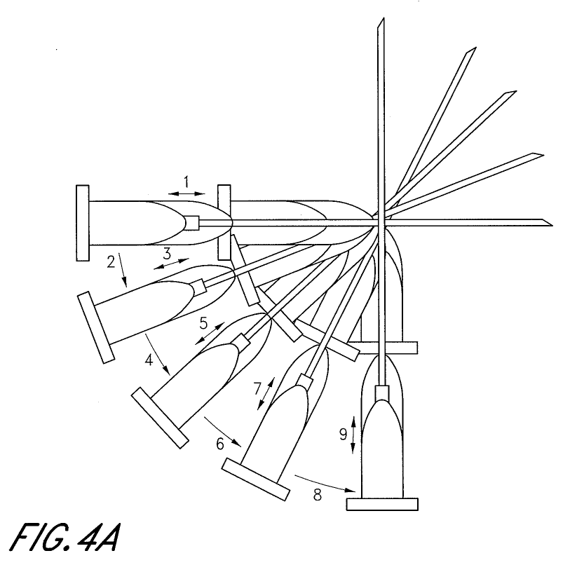

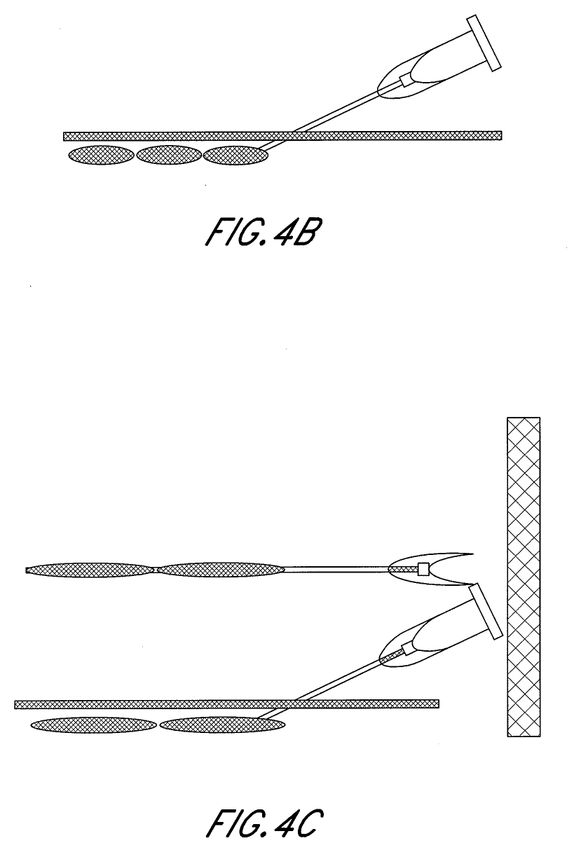

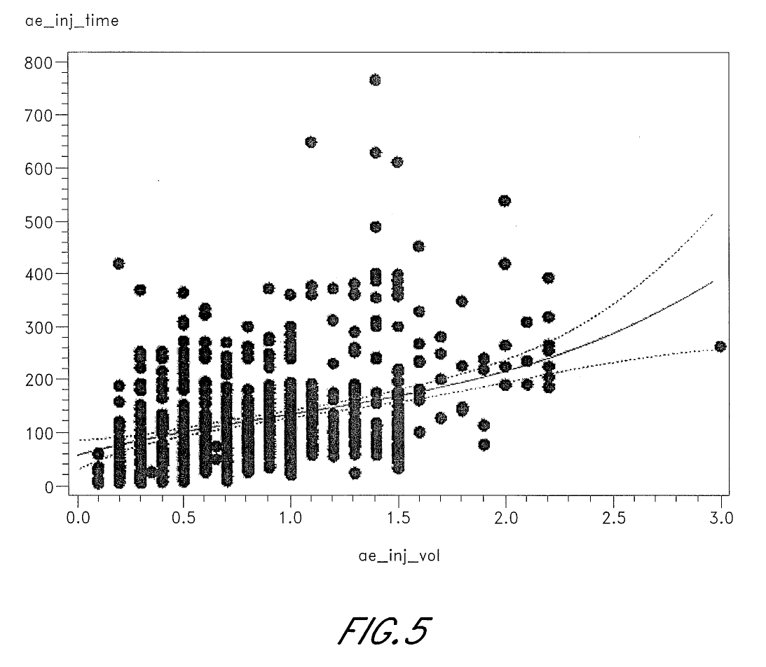

In addition to the recordation of the adverse events experienced by the subjects, data was also recorded on multiple variables, including the amount of filler injected, time taken for injection (by stopwatch), product injected, observed depth of injection (mid dermis, deep dermis or subcutaneous), calculated flow, number of injection sessions needed to achieve complete correction (number of touch-ups), and needle injection technique (e.g., fanning, multiple puncture, or linear threading). Exemplary embodiments of needle injection techniques used in the study are shown in FIGS. 4A-4C. FIG. 4A illustrates a fanning technique, FIG. 4B illustrates a multiple puncture technique, while FIG. 4C illustrates a linear/serial threading technique. Injection techniques are discussed in Matarasso S L, et al., “Consensus Recommendations for Soft-Tissue Augmentation with Nonanimal Stabilised Hyaluronic Acid (Restylane)” Plast. Reconstr. Surg. 117(3) Supplement: S3-34S (2006), which is hereby incorporated by reference in its entirety.

A statistical analysis of the data was used to identify whether an injection technique variable correlated with an increased rate of local adverse events. Logistic regression first identified some possible factors that could have been associated with adverse events. Data related to this logistic regression was collected and summarized in Table 1.0.

TABLE 1.0

| Factor | χ² | P |
|---|---|---|
| Injection time | 78.39 | <.0001 |
| Injection volume | 97.55 | <.0001 |
| Injection type | 52.91 | <.0001 |
| Injection depth | 0.52 | .7693 |
| Single injection sites | 0.11 | .7440 |

As shown in Table 1.0, the logistic regression identified possible factors associated with acute local adverse events. In contrast, other predefined variables were found to be unrelated to the rate of local adverse events, including the injection depth and injection site. The logistic regression suggested that adverse events were correlated with the following variables: injection type, fanlike injection technique, injection volume and correction time.

For those variables that showed a correlation with local adverse events, a subsequent sequential logistic regression was performed, while other variables were removed. The subsequent logistic regression revealed that the local adverse events remained strongly associated with total volume administered, rapid injection times, and fanlike injection technique.

With respect to the fanning technique, when comparing different types of injections, the data from the subsequent logistic regression revealed that 54% of fanlike injections produced adverse events compared with 20% for the other injection types combined, demonstrating that use of a fanning technique significantly increased the risk of adverse events. Table 1.1 below presents the pair-wise sequential logistic regression data for injection type versus adverse event.

TABLE 1.1

| Injection Technique | Estimate | P |
|---|---|---|
| Fanning vs. linear | 1.3594 | <.0001 |
| threading | | |
| Fanning vs. multiple | 1.6357 | <.0001 |
| puncture | | |
| Linear threading vs. | 0.2763 | .1527 |
| multiple puncture | | |

Similarly, volume had an estimate of 2.0063 (<0.0001) and time of minus 0.0107 (<0.0001) confirming their contribution to adverse events.

In regard to the volume of the filler used, FIG. 5 is a scatter plot that depicts the adverse events with respect to injection time versus injection volume. As the volume of dermal filler increases over time, the number of adverse events increases as well, as illustrated in FIG. 5 by the upward slope of the curves that track the change in number of adverse events.

With respect to the rapidity of the injection technique, it was demonstrated that the flow rate of the dermal filler had a strong correlation with the number of adverse events in a subject. Mean flow rates were estimated by dividing the total volume administered at one site by the total time to administer it. The data used to determine this is presented in FIG. 5. The data demonstrated that there was a dependency on the relationship between volume and time suggesting that the two variables were not independent. It was found that slow injection techniques resulted in decreased adverse events. In particular, as shown in FIG. 1, flow rates of greater than 0.3 mL/minute were shown to significantly increase adverse events, whereas flow rates of less than 0.8 mL/min. were shown to dramatically decrease the risk of an adverse event. Stated differently, flow rates of 0.3 mL/minute or less are associated with an extremely low incidence of adverse experiences. This finding was statistically significant. The 95% confidence intervals of the odds lie below one up until 0.85 mL/min when the interval widens (fewer observations).

Rapid injection and/or higher volume is associated with an increase in adverse events. Fanning injection technique is also associated with an increase in adverse events. Single-visit correction, years of physician experience and use of RESTYLANE® injectable filler versus PERLANE®) injectable filler had little or no impact on acute local adverse events. Further details regarding the above study and results can be found in “Effect of Injection Techniques on the Rate of Local Adverse Events in Patients Implanted with Nonanimal Hyaluronic Acid Gel Dermal Fillers,” Dermatologic Surgery, by Richard Glogau and Michael Kane, 34:S1 S105-S109, June 2008, incorporated in its entirety by reference.

In addition to the study data described above, data from an additional study was also examined to determine the mean and median flow rates for injections that resulted in adverse events and rejections that did not result in adverse events. This additional study, which may be referred to as Study A herein, was a randomized comparative evaluator-blinded study of the safety and efficacy of RESTYLANE® injectable filler and PERLANE® injectable filler in subjects with Fitzpatrick skin types 4, 5, or 6. The study discussed in greater detail above, which may be referred to herein as Study B, was a prospective, randomized, comparative, multi-center study of sensitization to RESTYLANE® injectable filler and PERLANE® injectable filler and included an acute safety profile assessment. The tables below summarize salient aspects of the data with respect to mean flow rates at various locations and how they correlated with the presence or absence of an adverse event. Table 1.2 and 1.3 are the results from the combination of the Study A and Study B. Tables 1.4 and 1.5 are the results from Study B only.

TABLE 1.2
Analysis Variable: inj_flow

| Ae | | | | | | | | |
|---|---|---|---|---|---|---|---|---|
| ind | wrinkle | N Obs | N | Mean | Minimum | Median | Maximum | Dev |
| No | Nasolabial fold | 649 | 649 | 0.6065079 | 0.0487805 | 0.5000000 | 2.0689655 | 0.4056934 |
| | Oral | 449 | 449 | 0.5791201 | 0.0638298 | 0.4313725 | 3.5000000 | 0.4953961 |
| | commissure | | | | | | | |
| Yes | Nasolabial fold | 217 | 217 | 0.7507159 | 0.1860465 | 0.6521739 | 2.5714286 | 0.4070139 |
| | Oral | 135 | 135 | 0.7597456 | 0.0983607 | 0.6428571 | 2.4000000 | 0.4265767 |
| | commissure | | | | | | | |

TABLE 1.3
Analysis Variable: inj_flow

| ae_ind | N Obs | N | Mean | Minimum | Median | Maximum | Dev |
|---|---|---|---|---|---|---|---|
| No | 1098 | 1098 | 0.5953083 | 0.0487805 | 0.4770333 | 3.5000000 | 0.4445543 |
| Yes | 352 | 352 | 0.7541790 | 0.0983607 | 0.6466063 | 2.5714286 | 0.4140450 |

TABLE 1.4
Analysis Variable: inj_flow

| Ae | | | | | | | | |
|---|---|---|---|---|---|---|---|---|
| ind | wrinkle | N Obs | N | Mean | Minimum | Median | Maximum | Dev |
| No | Nasolabial | 418 | 418 | 0.4940412 | 0.0487805 | 0.4148173 | 2.0689655 | 0.3474944 |
| | fold | | | | | | | |
| | Oral | 335 | 335 | 0.5285542 | 0.0638298 | 0.4000000 | 3.5000000 | 0.4701556 |
| | commissure | | | | | | | |
| Yes | Nasolabial | 148 | 148 | 0.8097830 | 0.2000000 | 0.6594203 | 2.5714286 | 0.4320237 |
| | fold | | | | | | | |
| | Oral | 120 | 120 | 0.7794264 | 0.2000000 | 0.6503497 | 2.4000000 | 0.4308006 |
| | commissure | | | | | | | |

TABLE 1.5
Analysis Variable: inj_flow

| ae_ind | N Obs | N | Mean | Minimum | Median | Maximum | Dev |
|---|---|---|---|---|---|---|---|
| No | 753 | 753 | 0.5093956 | 0.0487805 | 0.4067797 | 3.5000000 | 0.4067331 |
| Yes | 268 | 268 | 0.7961905 | 0.2000000 | 0.6533597 | 2.5714286 | 0.4309336 |

The “deviation” noted in the above tables denotes the variation of all of the observations of the flow rate.

The mean flow rate for all administrators in both studies A and B (n=1450) was about 0.634 mL/minute, the minimum flow rate was about 0.0488 mL/minute, the median flow rate was about 0.517 mL/minute, the maximum flow rate was 3.5 mL/minute, and the standard deviation was about 0.442 mL/minute. These values included the rates for those applications that resulted in no adverse events, and those applications that resulted in adverse events.

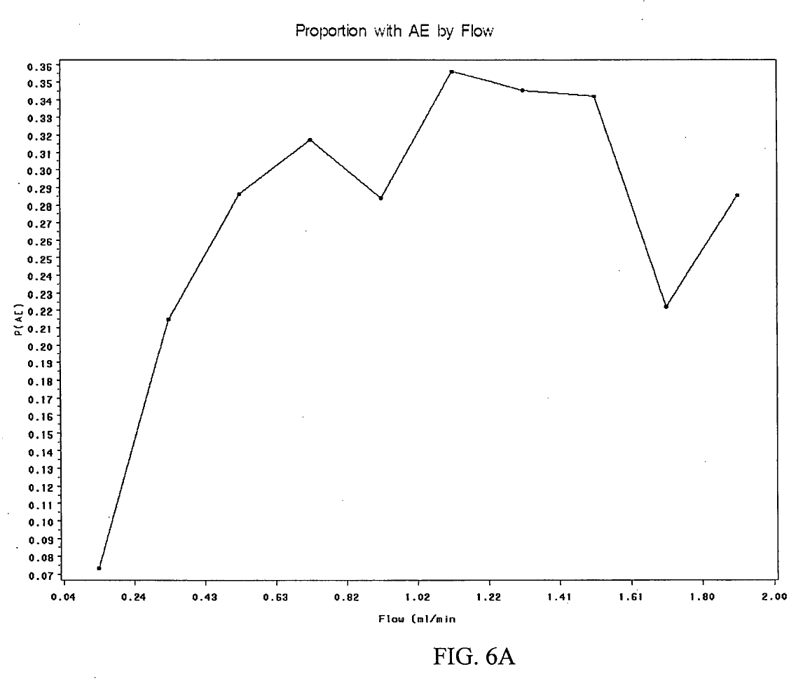

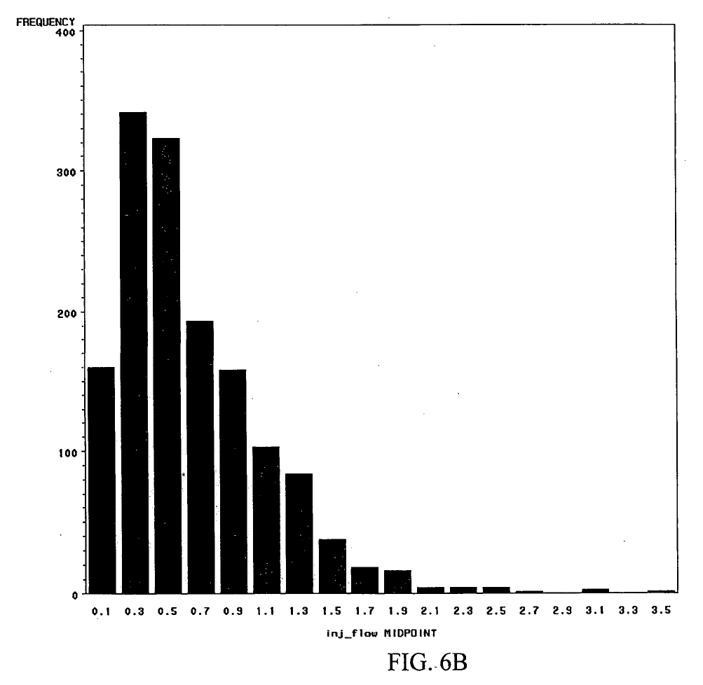

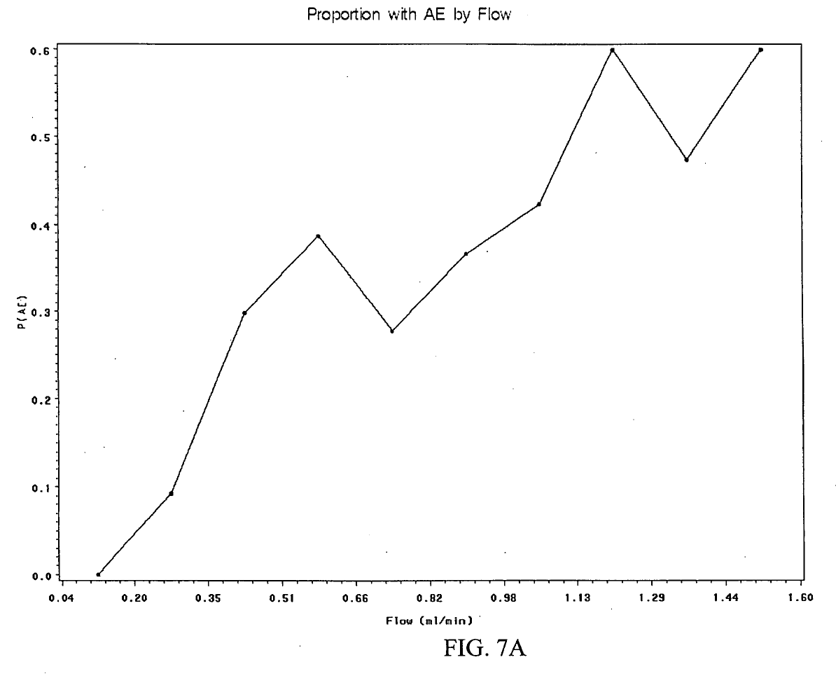

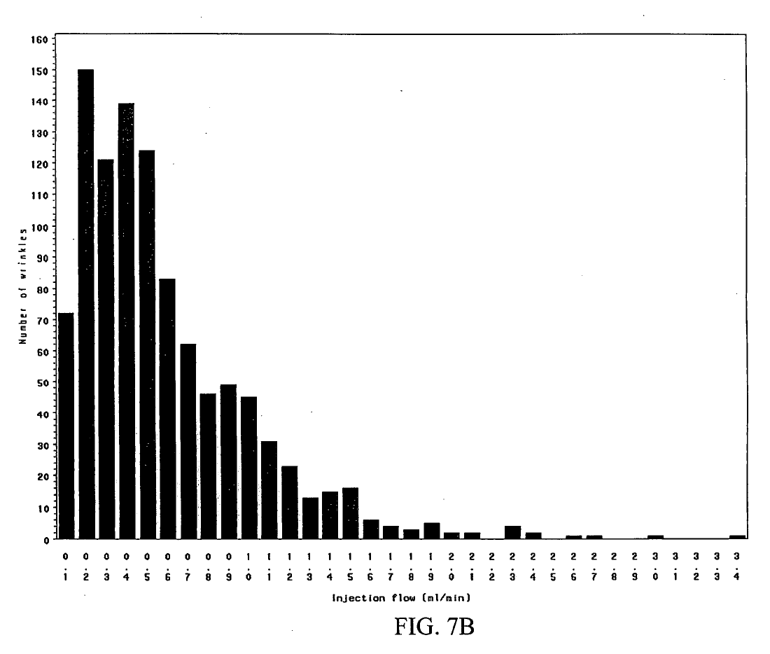

A further summary of the analysis can be found in FIGS. 6A, 6B, 7A, and 7B. FIGS. 6A and 6B display the results from the data from both study groups (i.e., the same combination as presented in Tables 1.2 and 1.3). FIGS. 7A and 7B display the results from the data from the single study group directed to this specific analysis (i.e., the same study as presented in Tables 1.4 and 1.5). FIGS. 6A and 7A display the probability that an adverse event will be experienced at a given flow rate. FIGS. 6B and 7B display the number of data points collected for each of the various flow rates.

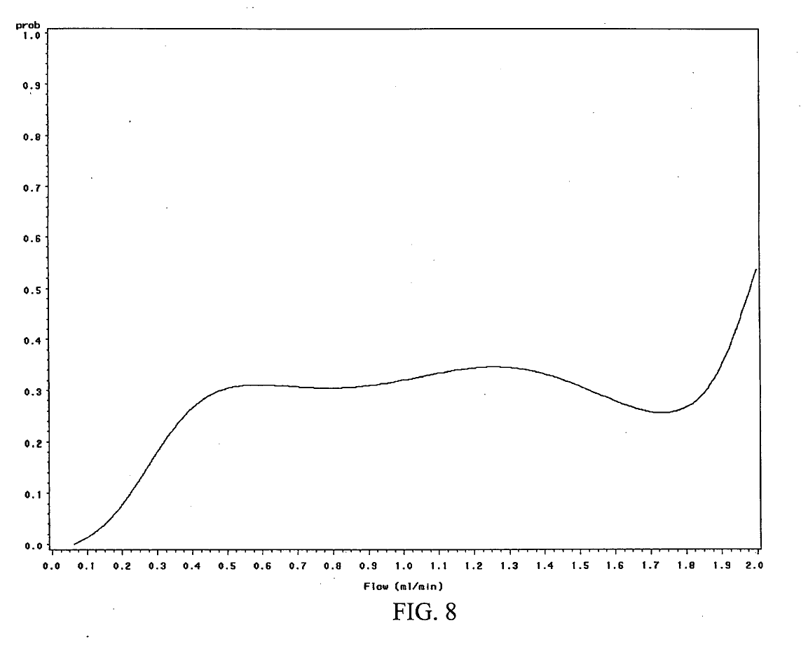

FIG. 8 is a graph that depicts the predicted probability of an adverse event occurring for injection volumes that were less than 2 mL. The graph includes the data from both studies A and B. The data in the figure were subjected to a high smoothing factor. As shown in the graph, significant reductions of the risk of adverse events clearly occur at 0.5 mL and lower. In addition, further significant reductions in the risk of an adverse event occur at flow rates of less than 0.35, such as at and less than 0.3 mL/minute. The y axis on FIG. 8 indicates the probability of an adverse event occurring. The statistical model used for the graph in FIG. 8 was a logistic regression model with injection flow specified as a 4^{th} degree polynomial effect.

### Example 1

A subject is injected with an injectable filler comprising a gel of non-animal stabilized hyaluronic acid (e.g., RESTYLANE® injectable filler). The treatment using the injectable filler is comprised of at least one injection to facial wrinkles around the nasal area. The injectable filler is injected using a linear threading technique. The mean flow rate of the injectable filler during the injection is maintained below 0.8 mL/min. The risk that the subject will experience bruising, redness, swelling, pain, tenderness, itching, pimples, firmness, lumps/bumps, discoloration, nodule formation, erythema, pruritus, desquamation, ecchymosis, edema, granuloma, contour irregularities, numbness, dryness, peeling, burning sensation, whiteheads, rash, and some combination thereof, at the treatment site, is thereby reduced.

### Example 2

A subject is injected with an injectable filler comprising a gel of biosynthesized, non-animal hyaluronic acid (e.g., JUVEDERM® injectable filler). The treatment using the injectable filler is comprised of at least one injection to facial wrinkles around the nasal area. The injectable filler is injected by using a linear threading technique. The flow rate of the injectable filler during the injection is maintained below 0.8 mL/min. The risk that the subject will experience an adverse event is thereby reduced.

### Example 3

A subject is injected with an injectable filler comprising polymethylmethacrylate (PMMA) microspheres suspended in a bovine culture (e.g., ARTEFILL™ injectable filler). The treatment using the injectable filler is comprised of at least one injection to facial wrinkles around the nasal area. The injectable filler is injected using a linear threading technique. The mean flow rate of the injectable filler during the injection is maintained below 0.8 mL/min. The risk that the subject will experience an adverse event is thereby reduced.

### Example 4

A subject is identified as having a history of adverse events due to the injection of an injectable fillers. The subject is injected with an injectable filler. The injectable filler is injected using a multiple puncture technique. For each treatment site treated with the injectable filler, the flow rate can be between 0.2 mL/minute and 0.6 mL/minute. The subject experiences a reduction in the risk of an adverse event occurring in the target area.

### Example 5

A subject identifies himself as desiring to minimize any risk of adverse events occurring at a treatment site. The treatment site is injected three times with a dermal filler. The mean flow rate during the first injection is 1 mL/minute. The mean flow rate during the second injection is 0.6 mL/minute. The mean flow rate during the third injection is 0.4 mL/minute. The risk that swelling, erythema, bruising, pain and/or tenderness will occur at the treatment site is reduced.

### Example 6

A subject is injected with an injectable filler comprising a gel of non-animal stabilized hyaluronic acid. The treatment using the injectable filler is comprised of at least one injection to facial wrinkles around the nasal area. The flow rate of the injectable filler during the injection is maintained below 0.8 mL/min. The severity of at least one of the following adverse events: bruising, redness, swelling, pain, tenderness, itching, pimples, firmness, lumps/bumps, discoloration, nodule formation, erythema, pruritus, desquamation, ecchymosis, edema, granuloma, contour irregularities, numbness, dryness, peeling, burning sensation, whiteheads, rash, and some combination thereof, at the treatment site, is thereby reduced.

### Example 7

An injectable filler is used throughout a treatment session of a subject and thereby restores volume and/or firmness to the subject's face. The injectable filler is injected by a linear threading technique and is not injected by a fanning technique.

For all of the injections of the injectable filler, the mean flow rate of each injection is beneath 0.8 mL/minute. However, the actual flow rate during each injection can vary. During at least one injection, the injectable filler is applied at a mean flow rate of 0.8 mL/minute; however, the initial half of the injection is applied at 0.6 mL/minute, while the second half of the injection is performed at 1 mL/minute. The risk that the subject will experience an adverse event is thereby reduced.

### Example 8

A subject will be injected with an injectable filler at one or more of the following locations: oral commissures, marionette lines, mandibular hollows, raised jowls, frowning mouth, pouty lower lips, lateral expression lines, mental creases, chin dimplings, zygomatic hollows, nasolabial folds, tear troughs, malar area or prominence, glabellar lines, crows feet, horizontal forehead lines, peri-oral vertical lines, and brow lifts. The subject will be treated using a non-fanning injection technique. The rate of injection will be less than 0.8 mL/minute. The risk that the subject will experience an adverse event is thereby reduced.

### Example 9

In order to determine whether or not an injectable filler can benefit from one or more of the methods disclosed herein, the injectable filler can be applied to two target areas on a subject. For the first target area, the injectable filler is applied at a flow rate of no more than 0.3 mL/min., using a linear threading technique, and applying no more than 1 mL of the injectable filler. For the second target area, the injectable filler is applied at a flow rate of at least 1 mL/minute, using a fanning technique, and applying at least 1 mL of the injectable filler. The process can be repeated for 10 target areas. The injectable fillers that result in more adverse events in the second target areas than the first target areas will be identified as acceptable injectable fillers.

In this disclosure, the use of the singular can include the plural unless specifically stated otherwise or unless, as will be understood by one of skill in the art in light of the present disclosure, the singular is the only functional embodiment. Thus, for example, “a” can mean more than one, and “one embodiment” can mean that the description applies to multiple embodiments. The phrase “and/or” denotes a shorthand way of indicating that the specific combination is contemplated in combination and, separately, in the alternative.

The section headings used herein are for organizational purposes only and are not to be construed as limiting the described subject matter in any way.

It will be appreciated that there is an implied “about” prior to the amounts, concentrations, times, etc. discussed in the present teachings, such that slight and insubstantial deviations are within the scope of the present teachings herein. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the invention.

The various devices and systems described above provide a number of ways to carry out the invention. It is to be understood that not necessarily all objectives or advantages described can be achieved in accordance with any particular embodiment described herein. Also, although the invention has been disclosed in the context of certain embodiments and examples, it will be understood by those skilled in the art that the invention extends beyond the specifically disclosed embodiments to other alternative embodiments and/or uses and obvious modifications and equivalents thereof. Accordingly, the invention is not intended to be limited by the specific disclosures of preferred embodiments herein.

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application; including but not limited to defined terms, term usage, described techniques, or the like, this application controls.

The foregoing description and Examples detail certain preferred embodiments of the invention and describes the best mode contemplated by the inventors. It will be appreciated, however, that no matter how detailed the foregoing can appear in text, the invention can be practiced in many ways and the invention should be construed in accordance with the appended claims and any equivalents thereof.

What is claimed is:

1. A method for reducing the risk that an adverse event will occur in a subject, the method comprising:
identifying a subject that will benefit from a reduction in the risk that an adverse event will occur due to the application of an injectable filler; and
injecting the subject with an injectable filler at a mean injection flow rate of less than 0.8 mL/minute.

2. The method of claim 1, with the proviso that a fanning technique is not employed for the injection.

3. A method for reducing the risk that an adverse event will occur in a subject, said method comprising applying an injectable filler to a subject, wherein an administrator of the injectable filler avoids administering the injectable filler at a mean flow rate of more than 0.8 mL/minute, and wherein the administrator avoids using a fanning injection technique.

4. The method of claim 3, wherein the adverse event is selected from the group consisting of: swelling, erythema, bruising, pain, and tenderness.

5. The method of claim 3, wherein the risk that an adverse event will occur is reduced to at least 10%.

6. The method of claim 3, wherein the method consists of:
applying the injectable filler to the subject via a multiple puncture and/or linear and/or serial threading injection technique; and
administering the injectable filler at a flow rate of no more than 0.3 mL/minute.

7. The method of claim 3, wherein the mean flow rate of the injectable filler is no greater than 0.63 mL/minute.

8. The method of claim 3, wherein the mean flow rate of the injectable filler is no greater than 0.3 mL/minute.

9. The method of claim 3, wherein the flow rate of the injectable filler is no more than 0.3 mL/min. for at least 50% of a time during which the injection occurs.

10. The method of claim 9, wherein the flow rate is no more than 0.3 mL/min. for at least 90% of the time during which the injection occurs.

11. The method of claim 9, wherein the flow rate is less than 0.048 mL/minute.

12. The method of claim 9, wherein the flow rate of the injectable filler is never greater than 0.63 mL/minute.

13. The method of claim 3, wherein the flow rate does not exceed 0.5 mL/minute throughout the application of the injectable filler, and wherein the injectable filler is applied by at least a first injection and a second injection.

14. The method of claim 13, wherein the injectable filler is applied by at least ten injections of the injectable filler.

15. The method of claim 13, wherein the injectable filler is applied by at least ten injections of the injectable filler, wherein for at least 90% of a time during which the at least ten injections of the injectable filler occur, the flow rate does not exceed 0.3 mL/min.

16. The method of claim 3, wherein the mean flow rate does not exceed 0.3 mL/min. throughout a treatment session.

17. The method of claim 3, wherein applying the injectable filler comprises each injection made during an entire treatment session, and wherein the flow rate does not exceed 0.5 mL/min. for at least 90% of a total time of all injections during the entire treatment session.

18. The method of claim 3, wherein applying the injectable filler to the subject is by an electronic device, wherein the electronic device ensures that the rate of injection is less than 0.4 mL/min.

19. The method of claim 3, wherein the injectable filler is selected from: non-animal stabilized hyaluronic acid, biosynthesized non-animal hyaluronic acid, polymethylmethacrylate microspheres suspended in bovine collagen, cross-linked hyaluronic acid from biofermentation, poly-L-lactic acid, microspheres of at least calcium and phosphate ions, and any combination thereof.

20. The method of claim 3, wherein a severity of at least one adverse event is reduced, the adverse event being selected from the group consisting of: swelling, erythema, bruising, pain, and tenderness.

21. The method of claim 3, wherein applying the injectable filler to the subject comprises providing an injectable filler volume that is a total of no more than 1 mL for a injection site.

22. The method of claim 3, wherein applying the injectable filler results in full correction in the subject.

23. The method of claim 3, wherein applying the injectable filler results in a decrease in a Wrinkle Severity Rating Scale (WSRS) by at least 1.

24. The method of claim 3, wherein the injectable filler is applied to an oral commissure, a nasolabial fold, or both.

25. A kit for reducing a risk that an adverse event will occur in a subject, comprising:
an injectable filler;
a set of instructions for administering the injectable filler, wherein the instructions provide that the injectable filler should be injected at a flow rate of no greater than 0.3 mL/minute; and
an injection device for applying the injectable filler to a subject.

26. The kit of claim 25, wherein the injectable filler comprises at least one injectable filler selected from the group consisting of: non-animal stabilized hyaluronic acid, biosynthesized non-animal hyaluronic acid, polymethylmethacrylate microspheres suspended in bovine collagen, cross-linked hyaluronic acid from biofermentation, poly-L-lactic acid, and microspheres of at least calcium and phosphate ions.

27. A method for reducing a risk of an adverse event occurring from the administration of an injectable filler, said method comprising providing instructions that the injectable filler should be applied at an injection rate of 0.8 mL/minute or slower, wherein the instructions are on a computer readable medium.

28. The method of claim 27, wherein the instructions are displayed on a website, and wherein the website is accessible to an injectable filler administrator.

29. The method of claim 27, wherein the instructions are recorded verbal instructions, and wherein the instructions are played over a phone line to an injectable filler administrator.

30. A method of distributing an injectable filler, said method comprising:
providing an injectable filler to an injectable filler administrator; and
providing a set of instructions, regarding how to apply the injectable filler, to the injectable filler administrator, wherein said set of instructions instruct the administrator to inject the injectable filler at a mean flow rate of no more than 0.6 mL/minute.

31. A method of reducing a rate of adverse events associated with an injectable filler in a population of subjects, said method comprising informing one or more injectable filler administrators that the injectable filler is to be injected at a mean flow rate of no more than 0.6 ml/min., wherein the population of subjects has the injectable filler injected by the one or more injectable filler administrators.

32. The method of claim 31, wherein the mean flow rate is no more than 0.3 mL/minute.

33. A method of treating a subject with an injectable filler, said method comprising:
identifying a subject that is to receive an injectable filler, wherein said subject desires to reduce a likelihood of any adverse events occurring from applying the injectable filler; and
applying the injectable filler to the subject, wherein the administrator selects a flow rate of the injection of the injectable filler such that a mean flow rate of the injectable filler is less than 0.5 mL/minute for all injections of the injectable filler in a treatment session.

34. A method of applying an injectable filler, said method comprising:
informing a subject to receive an injectable filler of two options, wherein a first option comprises a shorter treatment session and a higher risk of an adverse event, and wherein a second option comprises a longer treatment session and a lower risk of an adverse event;
receiving a selection of either the first or the second option from the subject;
and
applying the injectable filler to the subject, wherein if the subject selects the first option, the injectable filler is injected at a mean rate flow that is above 0.6 mL/min., and wherein if the subject selects the second option, the injectable filler is injected at a mean flow rate that is below 0.6 mL/min.

35. A method of applying an injectable filler to a subject, said method comprising:
injecting a subject with an injectable filler at a first rate;
recognizing an adverse event at an injection site on the subject; and
injecting the subject with the injectable filler at a second rate, wherein the second rate is slower than the first rate, and wherein the second rate is less than 0.8 mL/min.

36. A method of applying an injectable filler to a subject, said method comprising:
injecting a subject with an injectable filler at a flow rate that is less than 0.8 mL/min; and
informing the subject of the risk that they will experience an adverse event from the injection of the injectable filler, wherein the risk is no more than a risk associated with an injection of an injectable filler at a flow rate of no more than 0.8 mL/minute.

37. The method of claim 36, wherein the flow rate is no more than 0.3 mL/min.
